(19) United States
(12) Patent Application Publication
(10) Pub. No.: US 2026/0130651 A1
(43) Pub. Date: May 14, 2026

(54) Multi-Depth Ultrasound Imaging with a Multi-Frequency Probe

(71) Applicant: FUJIFILM SonoSite, Inc., Bothell, WA (US)

(72) Inventors: Ryan Hammond, Tacoma, WA (US); Jerome Mai, Seattle, WA (US); Jean Tsou, Seattle, WA (US); Bo Zhuang, Richmond, WA (US); Yong Zhou, Woodinville, WA (US)

(73) Assignee: FUJIFILM SonoSite, Inc., Bothell, WA (US)

(21) Appl. No.: 18/941,800

(22) Filed: Nov. 8, 2024

(51) Int. Cl. A61B 8/08; A61B 8/00

(52) U.S. Cl. CPC A61B 8/5246; A61B 8/4494; A61B 8/469; A61B 8/5207

(57) ABSTRACT

Systems and methods for multi-depth ultrasound imaging with a multi-frequency probe are described, which include a multi-array ultrasound scanner used to form a composite ultrasound image balancing image properties such as resolution, signal-to-noise ratio, and penetration. The multi-array ultrasound scanner includes two or more ultrasound transducer arrays capable of producing ultrasound radiation at two or more different frequencies configured to access two or more different depths in a subject. Two or more ultrasound signals with the two or more different frequencies are transmitted to a target, causing one or more return signals used to generate the composite ultrasound image. The ultrasound system can include a controller that implements uneven sampling so that regions outside a region of interest (ROI), far-field regions, regions without color when color imaging, and the like, are sampled less than more important regions, such as anatomies within an ROI, thus increasing a frame rate.

### BACKGROUND

Modern health care benefits from leveraging inter-body imaging, including techniques such as magnetic resonance imaging (MRI), computed tomography (CT), X-ray imaging, positron emission tomography (PET), and ultrasound. Ultrasound is a particularly feasible method of internal scanning as ultrasound systems can generate ultrasound images by transmitting sound waves at frequencies above the audible spectrum into a body, receiving echo signals caused by the sound waves reflecting from internal body parts, and converting the echo signals into electrical signals for image generation. Because they are non-invasive, non-ionizing, and real-time, ultrasound systems are used ubiquitously, such as in emergency medicine, critical care, and point of care. However, certain forms of imaging utilizing ionizing radiation, such as X-ray scans, typically have higher resolution than an ultrasound. Improvements in ultrasound technology, especially those increasing resolution and signal-to-noise ratio (SNR), are of great benefit to patients for ease of imaging, reduced side-effects over more invasive and/or more potentially harmful methods, correct diagnoses, etc.

Common emergency medicine and critical care procedures often require imaging with both high resolution and good penetration while maintaining sufficient SNR, contrast, and frame rate. High-frequency ultrasound probes, such as those operating above a threshold frequency value, can provide high-resolution images, but usually with the trade-off of having poor penetration. Similarly, low-frequency ultrasound probes, such as those operating below a threshold frequency value, usually provide good penetration, but with the trade-off of having poor resolution. Further, at large depths, the frame rate can be suppressed. A somewhat cumbersome solution is to employ several ultrasound probes and switch between them during an ultrasound examination. Accordingly, patients may be subjected to unnecessarily long examinations that may not provide the best imaging results.

### SUMMARY

Systems and methods for multi-depth ultrasound imaging with a multi-frequency probe are described, which include a multi-array ultrasound scanner used to form a composite ultrasound image balancing image properties such as resolution, signal-to-noise ratio, and penetration. The multi-array ultrasound scanner includes two or more ultrasound transducer arrays capable of producing ultrasound radiation at two or more different frequencies configured to access two or more different depths in a subject. Two or more ultrasound signals with the two or more different frequencies are transmitted to a target, causing one or more return signals used to generate a composite ultrasound image. The ultrasound system can include a controller that implements uneven sampling so that regions outside a region of interest (ROI), far-field regions, regions without color when color imaging, and the like, are sampled less than more important regions, such as anatomies within an ROI, thus increasing an effective frame rate.

In some implementations, an ultrasound device for multi-depth ultrasound imaging with a multi-frequency probe is disclosed. The ultrasound device includes a multi-array ultrasound scanner. The multi-array ultrasound scanner includes two or more transducer arrays, which are configured to generate two or more ultrasound signals including two or more different frequencies, the two or more different frequencies configured to access two or more different depths in a subject. The two or more transducer arrays are further configured to transmit the two or more ultrasound signals at the two or more different depths in an anatomy of the subject and receive a return signal based on the two or more ultrasound signals reflecting from the two or more different depths in the anatomy of the subject. The ultrasound device further includes one or more processors and a memory, the memory storing instructions that, when executed by the one or more processors, cause the one or more processors to generate an output. The output is based on the received return signal and the two or more different frequencies.

According to some examples, the return signal is a first return signal that is based on at least a first frequency of the two or more different frequencies. The two or more transducer arrays are further configured to receive a second return signal. The second return signal is based on the two or more ultrasound signals reflecting from the two or more different depths in the anatomy of the subject and at least a second frequency of the two or more different frequencies, the second frequency being different than the first frequency. In some examples, the output is a combination image, and the instructions further cause the one or more processors to generate, based on the first frequency, a first image data and generate, based on the second frequency, a second image data. A combination of the first image data and the second image data is used to generate the combination image.

In some implementations, a method for multi-depth ultrasound imaging with a multi-frequency probe is described. The method includes generating, by two or more transducer arrays of a multi-array ultrasound scanner, two or more ultrasound signals including two or more different frequencies, the two or more different frequencies configured to access two or more different depths in a subject. The method further includes transmitting, by the two or more transducer arrays, the two or more ultrasound signals at the two or more different depths in an anatomy of the subject and receiving, by the two or more transducer arrays, a return signal based on the two or more ultrasound signals reflecting from the two or more different depths in the anatomy of the subject. The method further includes generating, by one or more processors and based on the received return signal, an output, the output further based on the two or more different frequencies associated with the two or more ultrasound signals reflecting from the two or more different depths in the anatomy of the subject.

In some examples, the transmission of the two or more ultrasound signals includes one or more asymmetric transmission characteristics for the two or more ultrasound signals. According to some examples, the method further includes transmitting, by the two or more transducer arrays, at least one of the two or more ultrasound signals at an area of the subject outside of a region of interest (ROI) and receiving, by at least one of the two or more transducer arrays, an outside return signal based on the at least one of the two or more ultrasound signals reflecting from the area of the subject outside of the ROI. The output, in some examples, is further generated based on the outside return signal and further includes information corresponding to the at least one of the two or more ultrasound signals reflecting from the area of the subject outside of the ROI.

In some implementations, a user interface (UI) for multi-depth ultrasound imaging with a multi-frequency probe is disclosed. The UI is configured to register one or more user inputs, which cause one or more processors to generate, using two or more transducer arrays of a multi-array ultrasound scanner, two or more ultrasound signals using two or more different frequencies, the two or more different frequencies configured to access two or more different depths in a subject. The one or more user inputs further cause the one or more processors to transmit, using the two or more transducer arrays, the two or more ultrasound signals at the two or more different depths in an anatomy of the subject and receive, using the two or more transducer arrays, a return signal based on the two or more ultrasound signals reflecting from the two or more different depths in the anatomy of the subject. The one or more user inputs further cause the one or more processors to generate, based on the received return signal, an output, the output further based on the two or more different frequencies.

Other devices and methods to provide multi-depth ultrasound imaging with a multi-frequency probe are also described. These other devices and methods, in addition to those already disclosed, can be combined to generate additional devices and methods, which, though not explicitly disclosed herein, still are the same in concept as the devices and methods described explicitly herein. The devices and methods explicitly depicted herein are meant to be illustrative and not limiting.

### BRIEF DESCRIPTION OF THE DRAWINGS

The appended drawings illustrate examples and are, therefore, exemplary embodiments and not considered to be limiting in scope.

FIG. 1 illustrates an example environment for multi-depth ultrasound imaging with a multi-frequency probe.

FIG. 2 illustrates an example implementation of the ultrasound machine from FIG. 1.

FIG. 3 illustrates an example multi-array ultrasound transducer for multi-depth ultrasound imaging with a multi-frequency probe.

FIG. 4 illustrates an example of multi-frequency combinations for multi-depth ultrasound imaging with a multi-frequency probe.

FIG. 5 illustrates an example interleaving methodology for multi-depth ultrasound imaging with a multi-frequency probe.

FIG. 6 illustrates an example of multi-depth probing using multi-depth ultrasound imaging with a multi-frequency probe.

FIG. 7 illustrates an example of multi-beam (MB) probing using multi-depth ultrasound imaging with a multi-frequency probe.

FIG. 8 illustrates an example image of color flow within a color region in accordance with the present disclosure.

FIG. 9 illustrates an example image representing curved image data with coarser data in the far field than in the near field.

FIG. 10 illustrates an example diagram of adjusted beam angles to produce curved image data.

FIG. 11 illustrates an example of encoding and decoding for multi-depth ultrasound imaging with a multi-frequency probe.

FIG. 12 illustrates an example user interface for multi-depth ultrasound imaging with a multi-frequency probe.

FIG. 13 illustrates an additional example embodiment for the example user interface of FIG. 12.

FIG. 14 represents an example machine-learning architecture used to train a machine-learned model, which can be used to implement at least some of the techniques disclosed herein.

FIG. 15 represents an example model using a convolutional neural network (CNN) to process an input image.

FIG. 16 depicts a method for multi-depth ultrasound imaging with a multi-frequency probe.

FIG. 17 depicts a method for multi-depth ultrasound imaging with a multi-frequency probe.

FIG. 18 depicts a method for multi-depth ultrasound imaging with a multi-frequency probe.

FIG. 19 depicts a method for multi-depth ultrasound imaging with a multi-frequency probe.

FIG. 20 depicts a method for multi-depth ultrasound imaging with a multi-frequency probe.

FIG. 21 depicts a method for multi-depth ultrasound imaging with a multi-frequency probe.

FIG. 22 depicts a method for multi-depth ultrasound imaging with a multi-frequency probe.

### DETAILED DESCRIPTION

Common emergency medicine and critical-care procedures often require ultrasound imaging with both high resolution and good penetration while maintaining sufficient signal-to-noise ratio (SNR), contrast, and frame rate. High-frequency ultrasound probes can provide high-resolution images but usually have poor penetration. On the other hand, low-frequency ultrasound probes usually provide good penetration but have poor resolution. Further, at large depths, the frame rate can be slow. Hence, many users often purchase several ultrasound probes and switch between them during an ultrasound examination or, in a case where the user purchases a probe capable of multiple-frequency transmission, still perform multiple scans during the ultrasound examination. Accordingly, patients may be subjected to unnecessarily long examinations that may not provide the best imaging results. An ultrasound device leveraging the benefits of both high penetration and high resolution, while maintaining a high-level SNR, can greatly benefit the overall patient experience and health outcomes.

The present disclosure describes systems and techniques for multi-depth ultrasound imaging with a multi-frequency probe. The multi-frequency probe, in aspects, includes two or more transducer arrays, the two or more transducer arrays configured to transmit ultrasound radiation at an anatomy of a subject using two or more different frequencies. In aspects, the terms “multi-frequency probe” and “multi-array probe” are used interchangeably throughout this disclosure. As described above, different frequencies can have different characteristics, such as penetration power and resolution. By combining the two or more different frequencies in a same output image, both a good penetration and a high resolution can be realized with a single scan during an ultrasound examination. The ultrasound scanner includes two or more transducer arrays configured to transmit two or more ultrasound signals at two or more different frequencies. For example, the ultrasound scanner can have three transducer arrays and transmit three ultrasound signals using three different frequencies. In another example where the ultrasound scanner includes three transducer arrays, two of the transducer arrays can transmit two ultrasound signals at a first frequency and the third transducer array can transmit a third ultrasound signal at a second frequency. Other combinations of two or more transducer arrays transmitting two or more ultrasound signals at two or more different frequencies are also possible. In aspects, the two or more frequencies are configured to access two or more different depths in a subject.

The two or more ultrasound signals are transmitted by the two or more transducer arrays at an anatomy of the subject. The two or more ultrasound signals are reflected from the anatomy of the subject and are received by the two or more transducer arrays. For example, consider a multi-array ultrasound scanner including a first transducer array configured to transmit a first ultrasound signal at a first frequency and a second transducer array configured to transmit a second ultrasound signal at a second frequency, the second frequency being different than the first frequency. In one example, the first frequency can be transmitted at a first time and the second frequency can be transmitted at a second time, the second time being after the first time. A first return signal based on reflections of the first ultrasound signal from the anatomy of the subject can be received at a third time and a second return signal based on reflections of the second ultrasound signal from the anatomy of the subject can be received at a fourth time, the fourth time being after the third time. In one example, the first transducer array receives the first return signal and the second transducer array receives the second return signal.

In another example, again considering a multi-array ultrasound scanner including a first transducer array configured to transmit a first ultrasound signal at a first frequency and a second transducer array configured to transmit a second ultrasound signal at a second frequency, the second frequency being different than the first frequency, the first ultrasound signal and the second ultrasound signal can be transmitted at substantially a same time, for example, at a first time. A first return signal based on reflections of the first ultrasound signal from the anatomy of the subject and a second return signal based on reflections of the second ultrasound signal from the anatomy of the subject can be received at a second time. In some examples, the first return signal and the second return signal include a same combined return signal. In such examples, the combined signal can be received by the first transducer array, the second transducer array, or a combination of both the first transducer array and the second transducer array. In some examples, the first transducer array is configured to accept a first portion of the combined return signal that is based on the first frequency and the second transducer array is configured to accept a second portion of the combined return signal that is based on the second frequency. In some examples, the combined signal is analyzed by one or more processors to decompose the combined return signal into the first portion and the second portion, such as by a Fourier transform.

A return signal from the multi-array scanner (the first return signal and the second return signal, the combined return signal, etc.), in some examples, can be used by one or more processors of an ultrasound system to generate an output. For example, the output can be an image of a portion of the anatomy of the subject, the image containing information from both the first frequency and the second frequency. For example, the first frequency can be configured such that the first ultrasound signal provides good penetration but poor resolution. In this example, the second frequency can be configured such that the second ultrasound signal provides poor penetration but good resolution. In such an example, the image generated from the return signal based on the first frequency and the second frequency can include characteristics of both a high-penetration and a high-resolution image.

Additional embodiments and examples for multi-depth ultrasound imaging with a multi-frequency probe are provided herein. The examples depicted are meant to be illustrative and not limiting. Other valid combinations and embodiments can be derived from those depicted, and these other combinations and embodiments should be seen as implicitly disclosed herein.

### Example System

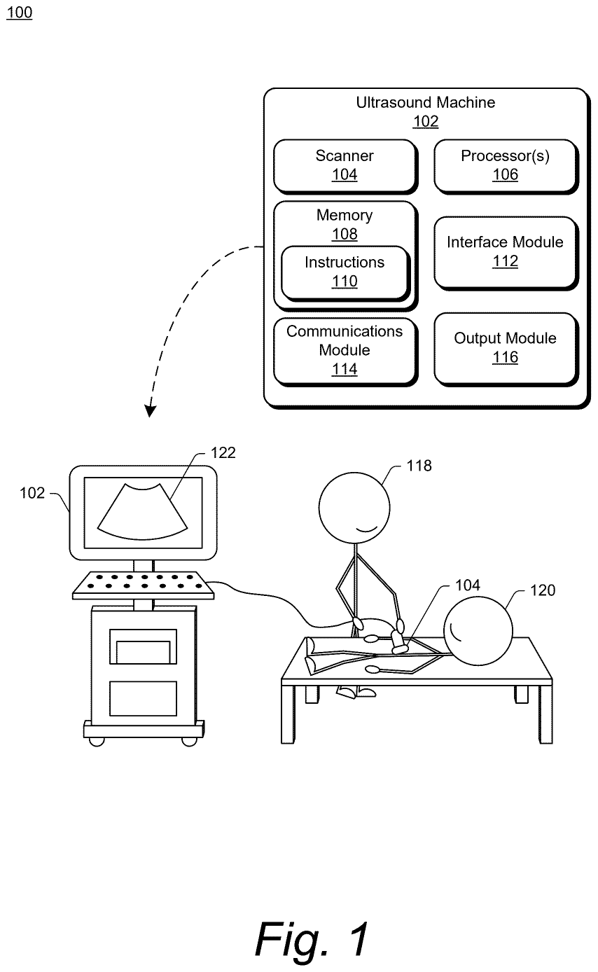

FIG. 1 illustrates an example environment 100 for an ultrasound machine 102 for multi-depth ultrasound imaging with a multi-frequency probe, in accordance with one or more implementations. Generally, the ultrasound machine 102 includes various components, some of which include a scanner 104, one or more processors 106, a memory 108 storing instructions 110, an interface module 112, a communications module 114, and an output module 116. In aspects, the scanner 104 is a multi-frequency scanner capable of outputting ultrasound radiation at multiple frequencies. Other components not illustrated may also be included. In some examples, the interface module 112 is a user input device (a keyboard, a cursor control device, a microphone, a camera, etc.). In some examples, the communications module 114 includes one or more wireless transmitters, receivers, or transceivers over a wireless connection or network (Bluetooth™, Wi-Fi™, etc.). In other examples, the communications module 114 includes a wired connection.

A user 118 (nurse, ultrasound technician, clinician, operator, sonographer, etc.) directs the scanner 104 toward a patient 120 to non-invasively scan internal bodily structures (anatomies, organs, tissues, etc.) of the patient 120 for testing, diagnostic, or therapeutic reasons. In some implementations, the scanner 104 includes two or more ultrasound transducer arrays and electronics communicatively coupled to the two or more ultrasound transducer arrays to transmit ultrasound signals to an anatomy of the patient 120 and receive ultrasound signals reflected from the anatomy of the patient 120 (e.g., echoes). In some implementations, the scanner 104 is an ultrasound scanner, which can be equivalently referred to as an ultrasound probe or transducer without distinction.

In aspects, the scanner 104 is configured to produce multi-depth ultrasound imaging with a multi-frequency probe, such as by generating a first ultrasound signal at a first frequency from a first transducer array of the ultrasound scanner 104 and a second ultrasound signal at a second frequency from a second transducer array of the ultrasound scanner 104. Although the disclosure herein considers the two-transducer-array embodiment with the first ultrasound signal at the first frequency and the second ultrasound signal at the second frequency, in general, the ultrasound scanner 104 includes two or more transducer arrays capable of producing two or more ultrasound signals at two or more different frequencies.

In some examples, the output module 116 is a display such as a display 122. The output module 116 is coupled to the one or more processors 106, which process the reflected ultrasound signals to generate ultrasound data. For example, consider the ultrasound scanner 104 including the two or more transducer arrays, where the two or more transducer arrays are capable of transmitting two or more ultrasound signals at two or more different frequencies. A return signal, for example, can be received based on reflections of the two or more ultrasound signals transmitted to the anatomy of the patient 120. The one or more processors 106 can, in some examples, generate an ultrasound image of the anatomy based on the return signal and the two or more different frequencies, which can be the ultrasound data. The output module 116 is, according to some examples, configured to generate and display the ultrasound image of the anatomy. In some examples, the ultrasound data includes the ultrasound image or data representing the ultrasound image.

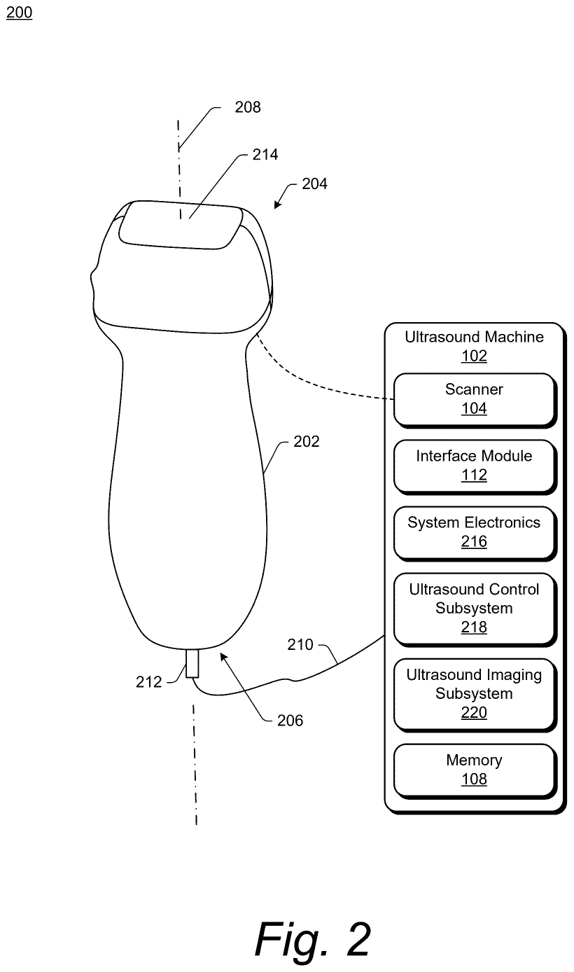

FIG. 2 illustrates an example implementation 200 of the ultrasound machine 102 from FIG. 1. The scanner 104 (e.g., an ultrasound scanner) includes an enclosure 202 extending between a distal end portion 204 and a proximal end portion 206. The enclosure 202 includes a central axis 208 (e.g., longitudinal axis) that intersects the distal end portion 204 and the proximal end portion 206. The central axis 208 corresponds to an axial direction of the scanner 104. In an example, the scanner 104 is electrically coupled to an ultrasound imaging system (e.g., the ultrasound machine 102) via a coupling 210. In implementations, the coupling 210 includes a cable that is attached to the proximal end portion 206 of the scanner 104 by a strain-relief element 212. In some implementations, the coupling 210 includes a wireless electronic coupling so that the scanner 104 is wirelessly coupled to the ultrasound imaging system and communicates with the ultrasound imaging system via one or more wireless transmitters, receivers, or transceivers over a wireless connection or network (Bluetooth™, Wi-Fi™, etc.).

A transducer assembly 214 (e.g., the scanner 104 of FIG. 1) having two or more transducer arrays is electrically coupled to system electronics 216 in the ultrasound machine 102. In operation, the transducer assembly 214 transmits ultrasound energy from the two or more transducer arrays toward a subject and receives ultrasound echoes from the subject. The ultrasound echoes are converted into electrical signals by the two or more transducer arrays and electrically transmitted to the system electronics 216 in the ultrasound machine 102 for processing and generation of one or more ultrasound images. In some examples, the transmitted ultrasound energy is in the form of ultrasound pulses. The ultrasound pulses can have parameters, such as waveform, phase, amplitude, target depth, and steering angle.

Capturing ultrasound data from a subject using a transducer assembly (the transducer assembly 214) generally includes generating ultrasound signals, transmitting ultrasound signals into the subject, and receiving ultrasound signals reflected by the subject. A wide range of frequencies of ultrasound can be used to capture ultrasound data, such as, for example, low-frequency ultrasound (e.g., less than a threshold megahertz (MHz) value) and/or high-frequency ultrasound (e.g., greater than the threshold MHz value). A particular frequency range to use can readily be determined based on various factors (depth of imaging, desired resolution, etc.). In some examples, the two or more transducer arrays of the transducer assembly 214 are configured to generate two or more ultrasound signals at two or more different frequencies. The two or more ultrasound signals can be transmitted at a same time, at different times, or at a combination of times (e.g., a first signal transmitted at a first time and both a second and a third signal transmitted at a second time). The two or more ultrasound signals can be configured to be transmitted at two or more different depths. In some implementations, the two or more transducer arrays of the transducer assembly 214 are configured to generate two or more ultrasound signals at a same ultrasound frequency.

In some implementations, the system electronics 216 include one or more processors (e.g., the processor(s) 106 from FIG. 1), integrated circuits, application-specific integrated circuits (ASICs), field programmable gate arrays (FPGAs), graphics processing units (GPUs), and power sources to support functioning of the ultrasound machine 102. In some implementations, the ultrasound machine 102 also includes an ultrasound control subsystem 218 having one or more processors. At least one processor, FPGA, ASIC, GPU, etc. causes electrical signals to be transmitted to the two or more transducer arrays of the transducer assembly 214 to both emit sound waves and also receive electrical pulses from the scanner 104 that were created from the returning echoes. One or more processors, FPGAs, ASICs, GPUs, etc. process the raw data associated with the received electrical pulses and form an image that is sent to an ultrasound imaging subsystem 220, which causes the image to be displayed (e.g., via an output module 116 of the ultrasound machine 102). In aspects, the output module 116 displays ultrasound images from the ultrasound data processed by the processor(s) of the ultrasound control subsystem 218.

In some implementations, the ultrasound machine 102 also includes one or more user input devices (a keyboard, a cursor control device, a microphone, a camera, etc.) that input data and enable taking measurements, such as from a display from the output module 116 of the ultrasound machine 102. The ultrasound machine 102 can also include a disk storage device (computer-readable storage media such as read-only memory (ROM), a Flash memory, a dynamic random-access memory (DRAM), a NOR memory, a static random-access memory (SRAM), a NAND memory, etc.) for storing the acquired ultrasound data. In aspects, the disk storage device includes a memory 108, which is local to the ultrasound machine 102. Alternatively, the memory 108 used for storing the acquisition data can be remote, such as on a remote server (e.g., medical archiver) communicatively connected to the ultrasound machine 102. In addition, the ultrasound machine 102 can include a printer that prints the image from the displayed data. To avoid obscuring the techniques described herein, some elements, such as user input devices, a disk storage device, and a printer, are not shown in FIG. 2.

### Example Multi-Array Scanning and Imaging

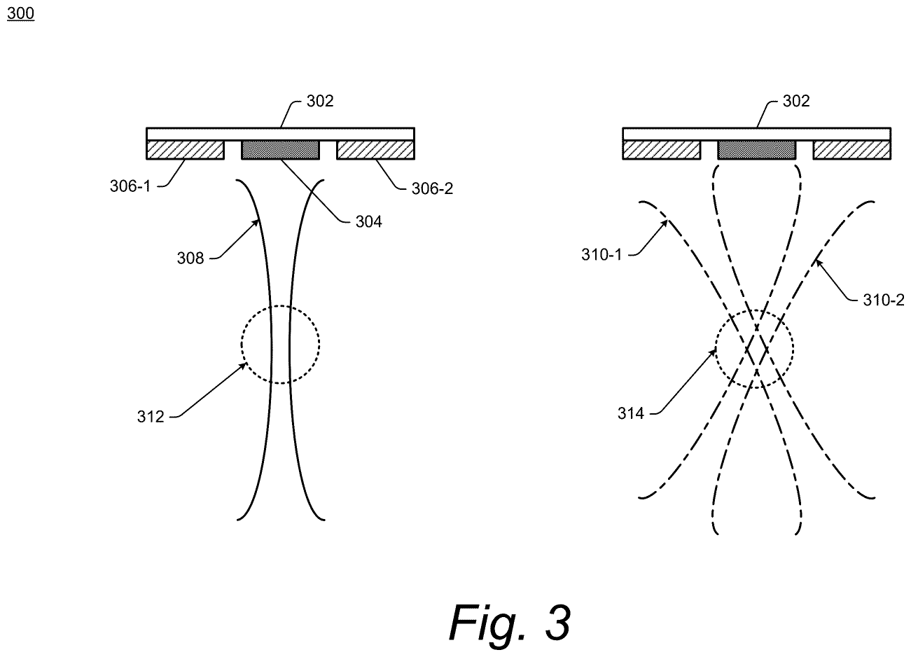

FIG. 3 illustrates an example multi-array ultrasound transducer 300 for multi-depth ultrasound imaging with a multi-frequency probe 302. For instance, a multi-array scanner in accordance with the present disclosure can include one or more of the arrays described in U.S. patent application Ser. No. 18/613,694 filed on Mar. 22, 2024, entitled Multi-Dimensional and Multi-Frequency Ultrasound Transducers to Zhang et al., the disclosure of which is incorporated herein by reference in its entirety. A multi-array scanner in accordance with the present disclosure can include one or more of the arrays described in U.S. patent application Ser. No. 17/561,313 filed on Dec. 23, 2021 entitled Array Architecture and Interconnection for Transducers to Li et al., the disclosure of which is incorporated herein by reference in its entirety.

The multi-frequency probe 302 includes a first array 304 and a second array 306. The second array 306 has a first sub-array 306-1 and a second sub-array 306-2. The first array 304 is configured to emit a first ultrasonic radiation with a first beam 308 at a first frequency (f₁), and the second array 306 is configured to emit a second ultrasonic radiation with a second beam pair 310 (with a second beam 310-1 and a third beam 310-2 from the first sub-array 306-1 and the second sub-array 306-2, respectively) at a second frequency (f₂). In aspects, the second frequency f₂ is different than the first frequency f₁ (f₁≠f₂); for example, f₁>f₂, f₁<f₂, etc.

The first array 304 and the second array 306 can operate in a same elevational plane. For example, the arrays 304 and 306 can be used to focus on a same point. Consider, for example, the first beam 308 having a beam pattern such that there is a focused portion 312 in the middle of the path illustrated in FIG. 3. The second beam 310-1 and the third beam 310-2, as illustrated, can be configured to emit such that their focused portions 314 coincide in space with the focused portion 312 of the first beam 308. This area of mutual focus can be, for example, at an anatomy of interest of a subject or a region of interest (ROI).

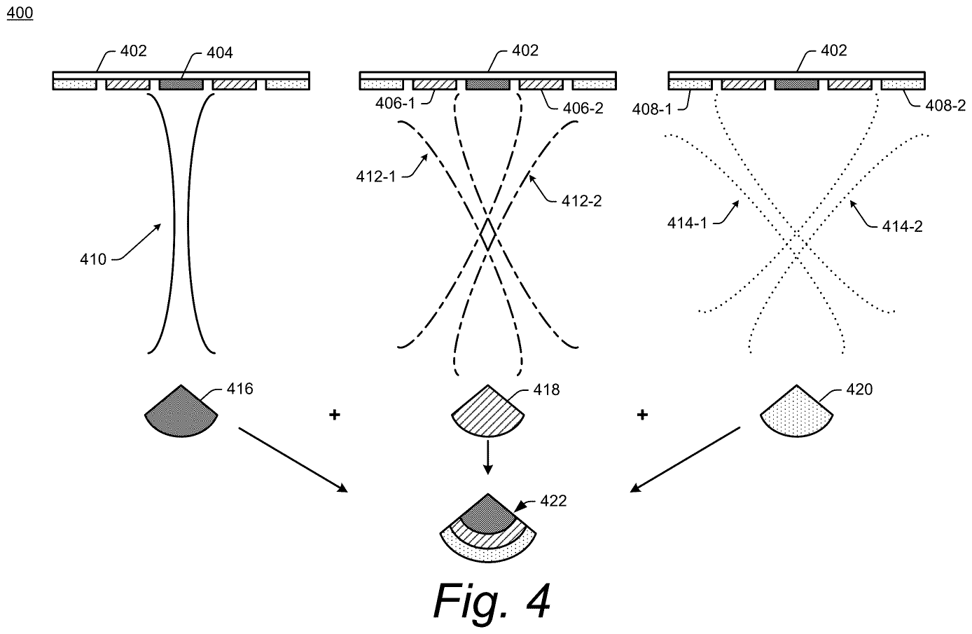

FIG. 4 illustrates an example of multi-frequency combinations 400 for multi-depth ultrasound imaging with a multi-frequency probe 402. An ultrasound system (e.g., the ultrasound machine 102 of FIG. 1) can use multiple arrays of a multi-array scanner, such as the multi-frequency probe 402, to form an image that balances imaging properties (e.g., resolution and penetration). For example, the multi-frequency probe 402 includes a first transducer array 404, which is configured to operate at a first frequency (f₁), a second transducer array 406 (with a first sub-array 406-1 and a second sub-array 406-2), which is configured to operate at a second frequency (f₂), and a third transducer array 408 (with a first sub-array 408-1 and a second sub-array 408-2), which is configured to operate at a third frequency (f₃). In this example, f₁/f₂/f₃. The first transducer array 404, in aspects, can produce a first ultrasound beam 410. The second transducer array 406, in aspects, can produce a second ultrasound beam pair 412 (with a second beam 412-1 produced by the first sub-array 406-1 of the second transducer array 406 and a third beam 412-2 produced by the second sub-array 406-2 of the second transducer array 406). The third transducer array 408, in aspects, can produce a third ultrasound beam pair 414 (with a fourth beam 414-1 produced by the first sub-array 408-1 of the third transducer array 408 and a fifth beam 414-2 produced by the second sub-array 408-2 of the third transducer array 408).

Consider, for example, f₃<f₂<f₁. Because of these frequencies, the beam width in an elevational plane for the third ultrasound beam pair 414 can be narrower than the beam widths for the first ultrasound beam 410 and the second ultrasound beam pair 412. Similarly, the beam width in the elevational plane for the second beam pair 412 can be narrower than the beam width for the first beam 410. For clarity, the differences in the beam widths are not illustrated in FIG. 4. As the first transducer array 404 has the highest operating frequency, it can have the highest resolution. By contrast, the relationship f₃<f₂<f₁ in the current example also means the first transducer array 404 has the poorest penetration characteristics. Similarly, in the present example, the third transducer array 408 can have the best penetration but the poorest resolution. The second transducer array 406 has a penetration and a resolution in between those of the first transducer array 404 and the third transducer array 408. Hence, to generate an ultrasound image that has both good resolution and good penetration, the ultrasound system can combine results from each of the three transducer arrays 404, 406, and 408 into a single image frame.

In aspects, the first transducer array 404 can contribute a first value 416, the second transducer array 408 can contribute a second value 418, and the third transducer array 408 can contribute a third value 420. A combined value 422 can be used to generate the single image frame. Various methods and techniques can be used to combine the values 416, 418, and 420 into the combined value 422. For instance, the first value 416 can be a first pixel value, the second value 418 can be a second pixel value, and the third value 420 can be a third pixel value, each of the pixel values derived and/or generated from one or more pixels based on data from the first beam 410, the second beam pair 412, and the third beam pair 414, respectively. In some examples, each of the beams 410, 412-1, 412-2, 414-1, and 414-2 can be used to generate discrete, corresponding images from which pixel data are extracted, with the first, second, and third pixel values based on one or more pixels from the corresponding images.

Consider, for example, a first image containing first pixels generated based on first data associated with the first beam 410, a second image containing second pixels generated based on second data associated with the second beam pair 412, and a third image containing third pixels generated based on third data associated with the third beam pair 414. The first value 416 can be pixel values from one or more pixels of the first image, the second value 418 can be pixel values from one or more pixels of the second image, and the third value 420 can be pixel values from one or more pixels of the third image.

The selection of the first value 416, the second value 418, and the third value 420, in some examples, can occur on a pixel-by-pixel basis or on a patch basis. For instance, a patch size of four-by-four pixels (16 pixels in total) can be selected from each of the three images. In other aspects, combining to generate the combined value 422 can include the blending of values (e.g., pixel values) from two or more of the values 416, 418, and 420. For instance, a new pixel value can be generated by taking X % from a pixel generated from one of the images and 100−X % from another pixel generated from another one of the images. In some examples, the images are not generated images but image data or arrays containing information on pixel values for rendering, where the combining can be done over any suitable number of the image data or arrays, including two, three, four, etc. In embodiments, the combining includes summing of values obtained from multiple image data or arrays. The summing can include averaging, generating a weighted average, etc. In some examples, the image data or arrays includes pre-scan-converted data.

In aspects, the system can combine data from the multiple image arrays based on a cost function of SNR, contrast, resolution, etc. One example of a cost function is:

$${\sum }_{i , l} {α}_{i , l} \cdot P [ {γ}_{l} ( {p}_{i} ) ] + β \qquad Eq . 1$$

In Eq. 1, the variable i indexes over the number of image arrays, the variable [symbol] indexes over the number of functions of a pixel (SNR, contrast ratio, confidence value, etc.), the operator γₗ denotes these functions, pᵢ denotes a pixel value for the i^{th} array, the operator P[⋅] is a patch operator that extracts pixels within a patch (and can, for example, sum their values), [symbol] is a programmable weight, and β is a bias term. The cost function can be optimized (minimized, maximized, etc.) over a choice of pixel functions, patch sizes, etc., to determine the pixel values that are used to populate corresponding values in a composite image, the composite image representing a combination of the images. In some examples, the composite image is a single image frame.

In some embodiments, the system implements a machine-learned model (e.g., a neural network) that has been trained to combine the first value 416, the second value 418, and the third value 420 into the combined value 422 for use in generating the composite image. In some examples, the machine-learned model can extract or otherwise generate the first value 416, the second value 418, and/or the third value 420 from ultrasound data generated by the multi-frequency probe 402. In some examples, the ultrasound system can provide the data from the three values 416, 418, and 420 to the machine-learned model, which generates the composite image, which can be displayed by the ultrasound system (e.g., on the display 122 of FIG. 1). In some examples, one or more features are first extracted from the data, such as with a convolutional neural network (CNN). Other aspects of machine learning related to multi-depth ultrasound imaging with a multi-frequency probe are discussed later in this disclosure.

In aspects, the system can start to generate an image frame before all the data is gathered from the transducer arrays 404, 406, and 408. For example, the ultrasound system can assemble pixels in the near field based on data from the first transducer array 404 while the system is still acquiring data in the far field from the third transducer array 408. In embodiments, the ultrasound system can cause the multi-frequency probe 402 to transmit ultrasound from multiple arrays (the first transducer array 404, the second transducer array 406, etc.) simultaneously. The frequencies can be selected to reduce interference, intermodulation, cross-talk, etc. For example, the ratio of any two of f₃, f₂, and f₁ can be an irrational number. In other examples, the frequencies can be selected as harmonics or subharmonics of one another. The harmonics and/or subharmonics can be selected, for instance, to support super harmonic imaging or sub-harmonic imaging.

In some aspects, the system combines data from the multi-frequency probe 402 across multiple image frames. For example, the system can combine the third value 420 based on data of the third transducer array 408 from a first previous image frame and the second value 418 based on data of the second transducer array 406 from a second previous image frame with the first value 416 based on data of the first transducer array 404 from a current image frame. This approach can reduce interference between the transducer arrays 404, 406, and 408 and reduce computational burden (e.g., compared to simultaneous use of the transducer arrays 404, 406, and 408) at the expense of delay/latency and/or tracking of time-varying data.

It should be noted that, though the previous discussion of combining data from the transducer arrays 404, 406, and 408 of FIG. 4 focuses on an embodiment using three arrays and three frequencies, this is meant to be illustrative and should not be construed as limiting. Any number of arrays may be used in any variety of configurations. For example, the first sub-array 408-1 of the third transducer array 408 can be a fourth transducer array configured to operate at a fourth frequency (f₄), with f₁≠f₂/f₃≠f₄, configured to operate at f₁, or any other configuration, which should be obvious to a skilled user.

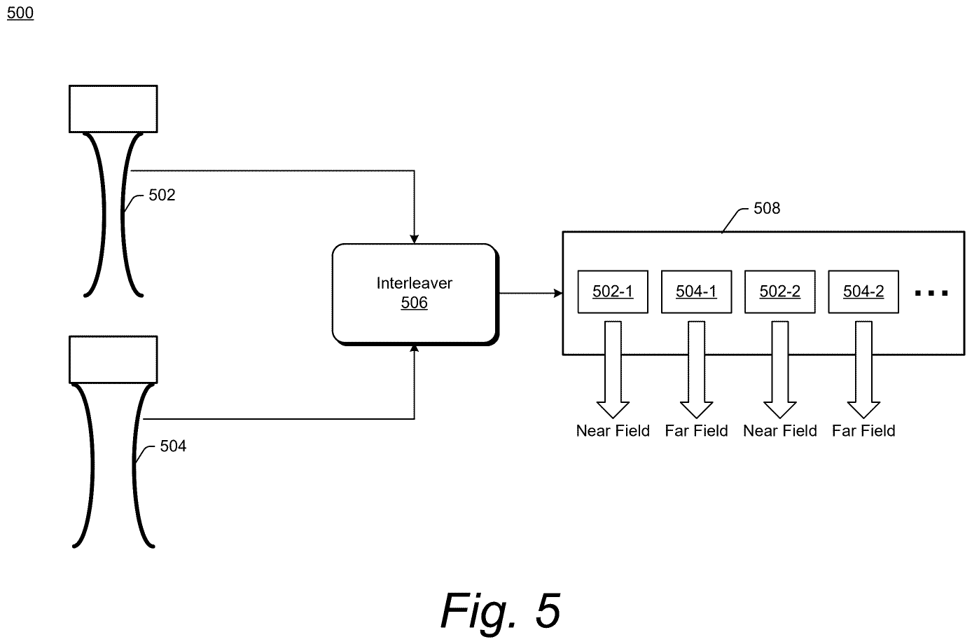

FIG. 5 illustrates an example interleaving methodology 500 for multi-depth ultrasound imaging with a multi-frequency probe. In some embodiments, an ultrasound system (e.g., the ultrasound machine 102 of FIG. 1) can use a single array, such as a single array of a multi-array scanner or a scanner that includes only a single array (e.g., the first transducer array 404 of FIG. 4). For example, a first ultrasound beam 502 can be generated with the array at a first frequency and a second ultrasound beam 504 can be generated with the array at a second frequency that is lower than the first frequency. In aspects and due to the frequency difference, the beam width of the first ultrasound beam 502 is narrower than the beam width of the second ultrasound beam 504.

An interleaver 506 interleaves copies of the first ultrasound beam 502 with copies of the second ultrasound beam 504 into an interleaved sequence 508. For example, instances 502-1 and 502-2 correspond to the first ultrasound beam 502, which can be used by the ultrasound system to generate image data in the near field. Additionally, in this example, instances 504-1 and 504-2 correspond to the second ultrasound beam 504, which can be used by the ultrasound system to generate image data in the far field. The ultrasound system can combine the near field and far field data as previously described in any suitable way.

Although the interleaving methodology 500 in FIG. 5 is described with respect to a single array, the interleaving methodology can also be implemented using multiple arrays. For instance, the first ultrasound beam 502 can be generated with a first array and the second ultrasound beam 504 can be generated with a second array. The first array and the second array can be part of a multi-array scanner (e.g., the multi-frequency probe 402 of FIG. 4). In another example, the first array can be included in a first ultrasound scanner, and the second array can be included in a second ultrasound scanner.

In some examples, the frame rate can be reduced due to the use of multiple ultrasound pings to construct a single image frame (e.g., a composite image). To recover some of the frame rate, in aspects, an ultrasound system (e.g., the ultrasound machine 102 of FIG. 1) can employ uneven sampling to data gathered in regions that are not an ROI, such as regions outside the ROI, far field regions, regions without color when color imaging, etc., compared to the ROI or another region.

In aspects, a multi-frequency probe in accordance with the present disclosure can configure its different arrays to operate in different imaging modes. For example, the first transducer array 404 can be configured to operate in a first imaging mode, and the second transducer array 408 can be configured to operate in a second imaging mode. Examples of different imaging modes include B-mode imaging, C-mode imaging, M-mode imaging, color Doppler imaging, and the like. A low-frequency array can be implemented to image in B-mode and a high-frequency array can be implemented to image in a Doppler mode. The system can configure the multiple arrays in any suitable combination of imaging modes, using any suitable ultrasound frequencies. The arrays can be configured to operate at different frequencies or at a same frequency. Image data from the multiple arrays operating in different imaging modes can be interleaved by the interleaver 506, and the display 122 can simultaneously display two ultrasound images, such as a B-mode image and a color Doppler image generated from the different arrays. The display can appear to a user without perceptible delay, in real time.

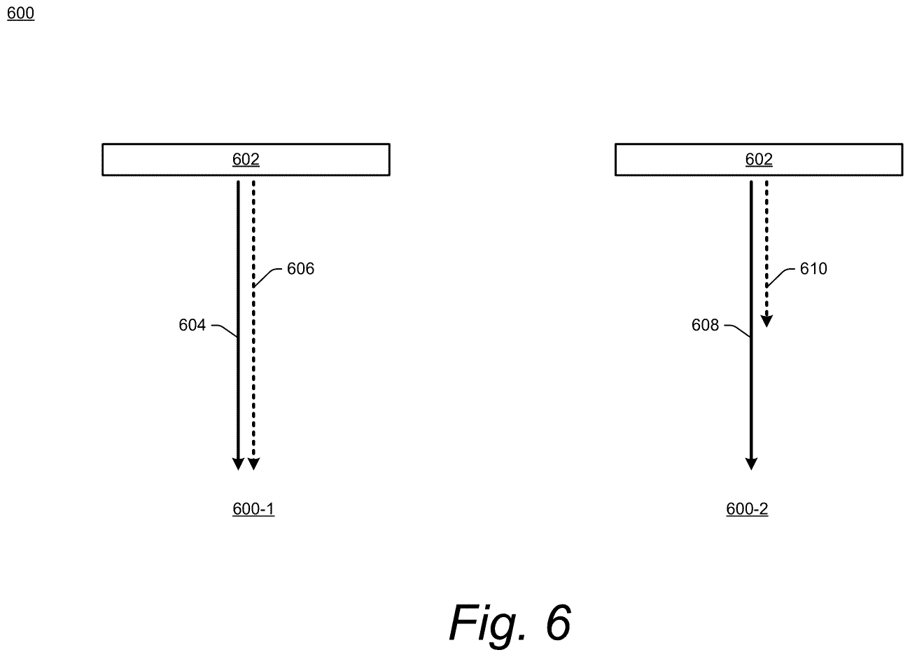

FIG. 6 illustrates an example 600 of multi-depth probing for multi-depth ultrasound imaging with a multi-frequency probe 602. Pulse acquisition is based on depth, in accordance with the present disclosure. In the left diagram 600-1 of FIG. 6, the multi-frequency probe 602 emits a first pulse 604 and a second pulse 606. The first pulse 604 and the second pulse 606 are of the same depth. For example, the first pulse 604 can be transmitted by a first array of the multi-frequency probe 602 at a first frequency (f₁), such as the first array 304 of FIG. 3, and the second pulse 606 can be transmitted by a second array of the multi-frequency probe 602 at a second frequency (f₂), such as the first sub-array 306-1 of the second array 306 of FIG. 3. In aspects, an ultrasound system (e.g., the ultrasound machine 102 of FIG. 1) can use the first pulse 604 for far field imaging and the second pulse 606 for near field imaging.

In an example where the second pulse 606 is used for near field imaging and not for far field imaging, there is little reason to collect far field data from the second pulse 606. For example, f₂ can be selected for a desired resolution in near field imaging and thus can be less suited for a penetration characteristic desired for far field imaging. In aspects, the ultrasound system can shorten the acquisition time (and hence the depth) for the second pulse 606, thereby increasing the frame rate achieved by the system.

In the right diagram 600-2 of FIG. 6, the multi-frequency probe 602 emits a third pulse 608 at f₁ and a fourth pulse 610 at f₂. The third pulse 608 and the fourth pulse 610 are of different depths. In examples, the ultrasound system can generate an ultrasound image with parts that are collected at different frame rates. First, the ultrasound system can collect data corresponding to deeper portions of the image, such as using the third pulse 608, less frequently. This can have the effect of saving overall time, using fewer resources of the ultrasound device, etc. Second, the ultrasound system can collect data corresponding to shallower portions of the image, such as using the fourth pulse 610, more frequently. This can have the effect of increasing the frame rate of the shallower portions. In order to generate images including both near field and far field data acquired at different rates, the ultrasound system can re-use previous frame data at deeper depths. For example, data for a deeper depth (e.g., far field data) can be obtained from a previous image frame and used in a current image frame.

In examples using uneven sampling, an ROI in an anatomy of a subject is defined, such as an area of an ultrasound image of the anatomy of the subject. For instance, the ROI can be defined by a user, generated by a machine-learned model, part of a default range of an ultrasound device, etc. In an example, a neural network generates the ROI and the user grabs and drags handle locations on an interface displaying at least the ROI to deform the ROI into a desired boundary that encompasses a portion of the anatomy of the subject the user wishes to image. For areas that are outside the ROI, the system can suppress ultrasound pings and therefore increase an effective frame rate.

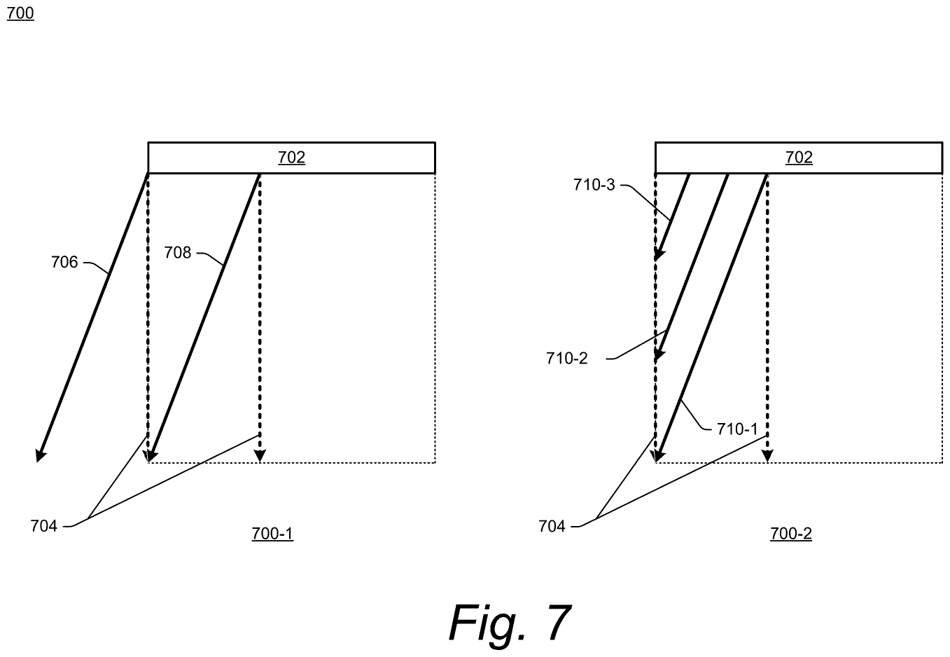

FIG. 7 illustrates an example of multi-beam (MB) probing 700 using multi-depth ultrasound imaging with a multi-frequency probe 702. A left-side image 700-1 of FIG. 7 has an ROI indicated by dashed lines 704. The ROI can be determined by a machine-learned (ML) model, a user selection, or any other means of determining the ROI. The multi-frequency probe 702 emits a first ultrasound beam 706 and a second ultrasound beam 708. The second ultrasound beam 708 is within the ROI and the first ultrasound beam 706 is outside of the ROI. In some examples, the multi-frequency probe 702 can limit the output of the first ultrasound beam (lower the acquisition rate, not transmit the first ultrasound beam 706, lower a power of transmission of the first ultrasound beam 706, etc.).

In accordance with some embodiments disclosed herein, however, the multi-frequency probe 702 can limit transmission of ultrasonic radiation to only that which will be incident upon the ROI, the multi-frequency probe 702 can only transmit ultrasonic radiation that will be incident upon the ROI. In a right-side image 700-2 of FIG. 7, the multi-frequency probe 702 transmits multiple beams 710. As illustrated, individual beams 710-1, 710-2, and 710-3 are all incident in the ROI, which is indicated by the dashed lines 704. By limiting output to only parts of the multi-frequency probe 702 (e.g., transducer arrays that can point at the ROI), in some examples, resources of an ultrasound device (e.g., the ultrasound machine 102 of FIG. 1) can be conserved or otherwise optimized.

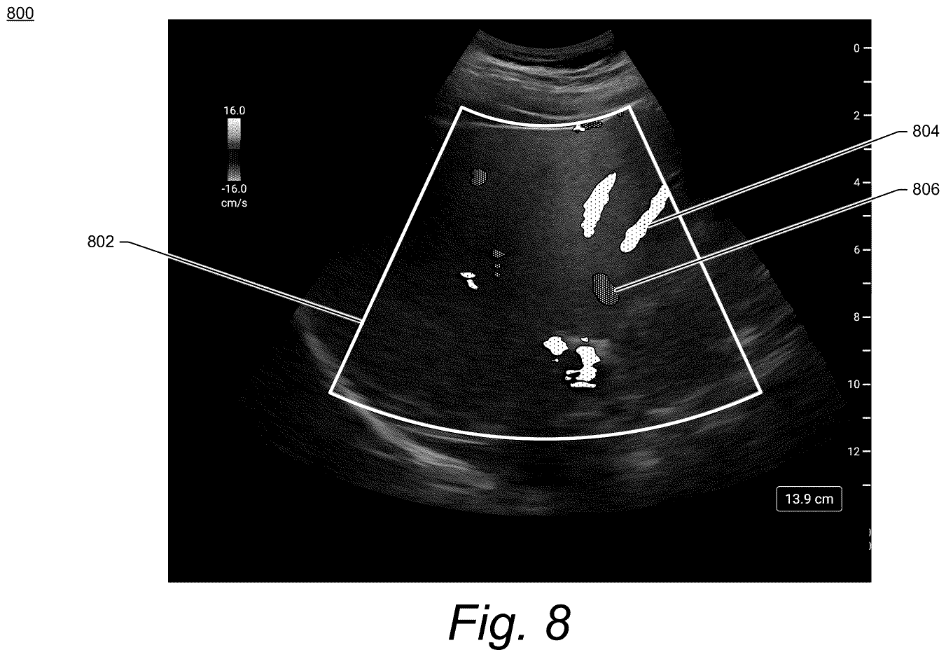

FIG. 8 illustrates an example image 800 of color flow within a color region 802 in accordance with the present disclosure. When color imaging is used, such as to visually represent fluid flow in an image of an anatomy of a subject, an ultrasound system (e.g., the ultrasound machine 102 of FIG. 1) can restrict a transmission of ultrasound radiation and acquisition of data based on the ultrasound radiation to regions that are inside the color region 802. The color region 802 renders a first color flow 804 and a second color flow 806. The first color flow 804 and the second color flow 806 can, in some examples, represent a fluid flow, such as a blood flow. The color region 802 (here shown as trapezoidal in shape, though other shapes may be used) is an example of an ROI, as previously described. As such, in examples, the ultrasound system can restrict the transmission of the ultrasound radiation and acquisition of the data based on the ultrasound radiation to the ROI to save resources, increase the frame rate, etc. Additionally or alternatively, the ultrasound system can restrict transmission of the ultrasound radiation and acquisition of the data based on the ultrasound radiation to portions of the color region 802 that have color, such as the first color flow 804 and the second color flow 806. In aspects, the system can define the ROI as the union of regions that contain color.

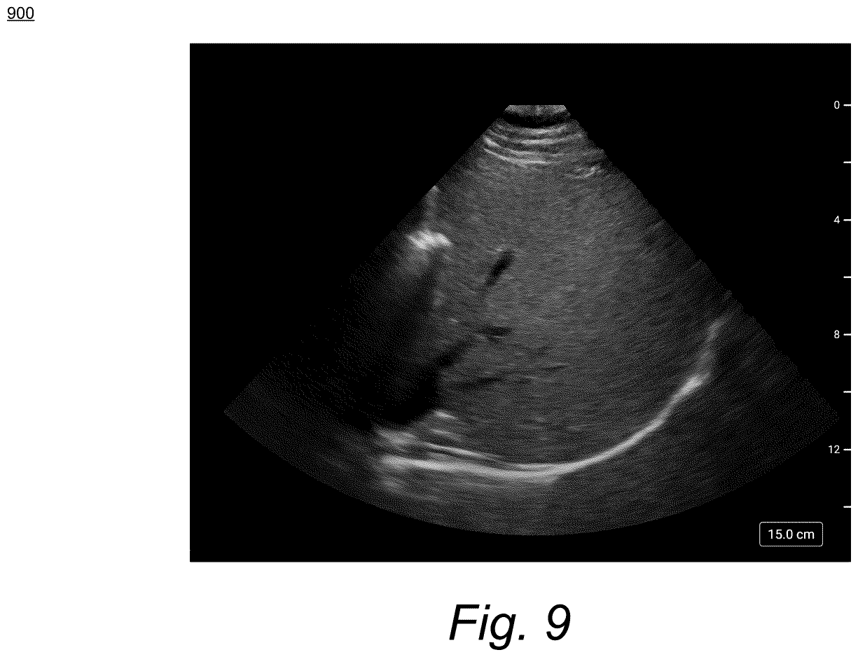

FIG. 9 illustrates an example image 900 representing curved image data with coarser data in the far field than in the near field. For curved imaging formats, such as when using a curvilinear array, a resulting ultrasound image, such as the image 900, is often coarser in the far field than in the near field. Therefore, in some embodiments, an ultrasound system (e.g., the ultrasound machine 102 of FIG. 1) can implement uneven sampling to reduce the number of transmissions and acquisitions in the near field relative to the far field. In embodiments, this approach achieves evenly distributed spatial sampling across near and far fields.

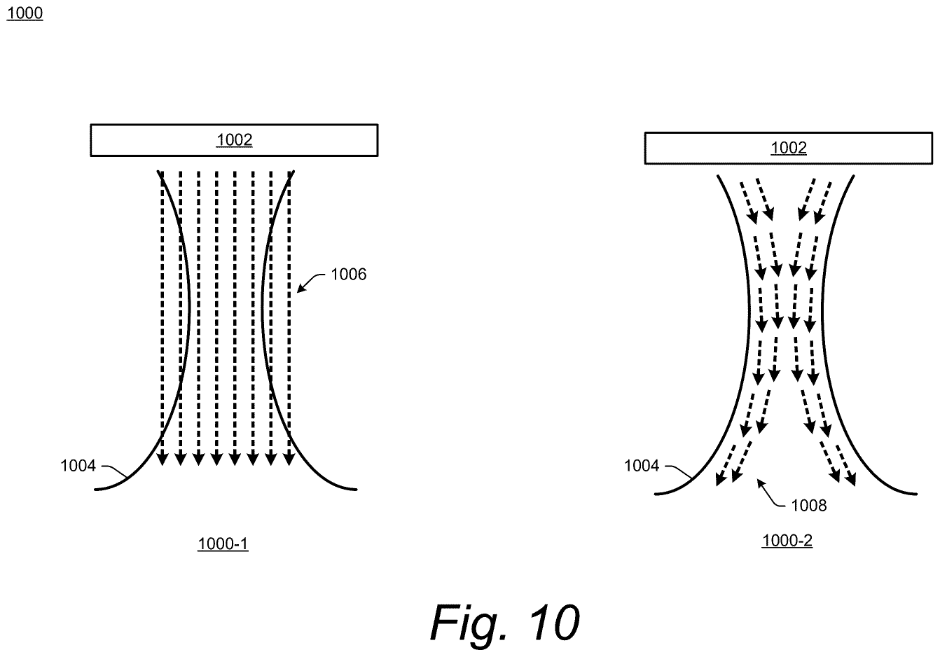

FIG. 10 illustrates an example diagram 1000 of adjusted beam angles to produce curved image data. In some embodiments, an ultrasound system (e.g., the ultrasound machine 102 of FIG. 1) can use a multi-frequency scanner 1002 to adjust beam angles and frequencies so that ultrasound signals are transmitted such that they follow an ultrasound field 1004, rather than simply being transmitted in a straight or linear fashion. Consider, as an example, a left-side image 1000-1 of FIG. 10. The multi-frequency scanner 1002 transmits first signals 1006 in a linear fashion. It can be seen that the first signals 1006 do not follow the shape of the ultrasound field 1004.

Consider, for example, a right-side image 1000-2 of FIG. 10. The multi-frequency scanner 1002 transmits second signals 1008 in a non-linear fashion, such as by the multi-frequency array 1002 steering ultrasound transducer arrays and/or steering emitted beams of the ultrasound transducer arrays. This beam steering, in some examples, results in the second signals 1008 following more closely the shape of the ultrasound field 1004. The beam steering, in some examples, is produced by the multi-frequency scanner 1002 physically aiming the transducer arrays in one or more directions, which can be one or more different directions. In some examples, the second signals 1008 are the product of one or more beam combinations, the one or more beam combinations configured such that a resulting ultrasound transmission gains the shape of the second signals 1008. In some examples where the second signals 1008 are the product of the one or more beam combinations, the transducer arrays of the multi-frequency scanner 1002 can be configured to emit/transmit ultrasound radiation in a linear fashion. The ultrasound system, in some examples, can maximize the energy usage and the resulting SNR by producing ultrasound signals that follow the ultrasound field 1004 more closely, such as the second signals 1008. These features can be combined with any of the features previously described to generate an ultrasound image efficiently and with high SNR.

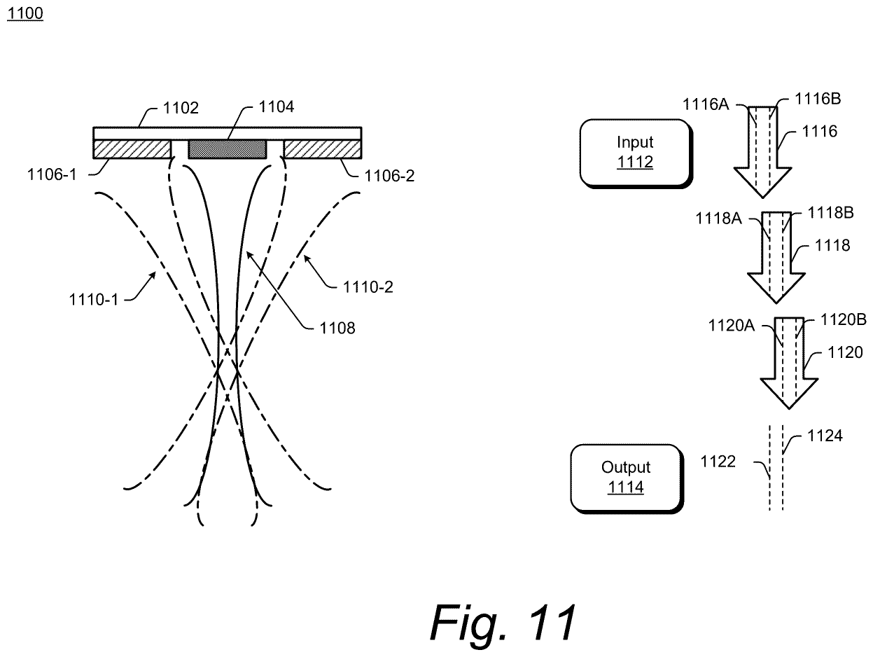

FIG. 11 illustrates an example 1100 of encoding and decoding for multi-depth ultrasound imaging with a multi-frequency probe 1102. An example of encoding and decoding ultrasound signals for ultrasound imaging is described in U.S. patent application Ser. No. 18/593,339 filed on Mar. 1, 2024, entitled “Multi-Mode Rolling-Encoded Ultrasound” to Zhou et al., the disclosure of which is incorporated by reference herein in its entirety. The multi-frequency probe 1102 includes multiple transducer array components, such as a first array 1104 and a second array 1106, which includes a first sub-array 1106-1 and a second sub-array 1106-2. In this embodiment, the multi-frequency probe 1102 (e.g., the scanner 104 of FIG. 1) can take advantage of a broader transmission and multi-line reception. For each of these two or more transmissions, such as transmissions 1108 and 1110 (with a first component 1110-1 and a second component 1110-2 from the first sub-array 1106-1 and the second sub-array 1106-2, respectively), in aspects, there can be two or more corresponding receptions and radio-frequency (RF) data. In some examples, there can be a series of pings sent out with multi-mode encoding, such as using dual mode type or mode variant pings, though other mode types or variants within a mode type can equivalently be used.

An ultrasound system (e.g., the ultrasound machine 102 of FIG. 1) can receive multi-mode ping-based data as inputs 1112 and combine the inputs 1112 as outputs 1114. For example, a first multi-mode ping 1116 can be sent with a first mode 1116A and a second mode 1116B. A second multi-mode ping 1118 can be sent with a third mode 1118A and a fourth mode 1118B. A third multi-mode ping 1120 can be sent with a fifth mode 1120A and a sixth mode 1120B. Note that modes 1116B and 1118A overlap in a first point in space 1122 and modes 1118B and 1120A overlap in a second point in space 1124.

Spatial rolling encoding can be realized using, in a non-limiting example, a bipolar Hadamard matrix encoding and decoding, and the modes 1116A, 1116B, 1118A, 1118B, 1120A, and 1120B can use multi-mode types. A first mode type can, in some examples, correspond with received RF pings represented by RF₁, and a second mode type can correspond with received RF pings represented by RF₂. The encoding in such an example, when using a bipolar Hadamard matrix encoding, has the following representation:

$$1116 B = R {F}_{1} + R {F}_{2} \qquad Eq . 2$$

$$1118 A = R {F}_{1} - R {F}_{2} \qquad Eq . 3$$

$$1118 B = R {F}_{1} - R {F}_{2} \qquad Eq . 4$$

$$1120 A = R {F}_{1} + R {F}_{2} \qquad Eq . 5$$

Reception of these modes RF₁ and RF₂ can be recovered from signals received as a multi-mode reception signal, such as those originating at the points in space 1122 and 1124. In aspects, the point in space 1122 represents a region of space where the modes 1116B and 1118A are incident and the point in space 1124 represents a region of space where the modes 1118B and 1120A are incident. For example, taking the polarities of Eqs. 2-5 results in the following equations:

$$R {F}_{1} = ( 1 \; 116 B + 1 \; 118 A ) / 2 \qquad Eq . 6$$

$$R {F}_{2} = ( 1 \; 116 B - 1 \; 118 A ) / 2 \qquad Eq . 7$$

$$R {F}_{1} = ( 1 \; 118 B + 1 \; 120 A ) / 2 \qquad Eq . 8$$

$$R {F}_{2} = ( 1 \; 120 A - 1 \; 118 B ) / 2 \qquad Eq . 9$$

According to the illustrated example 1100, Eqs. 6 and 7 are using data from signals originating at the point in space 1122 and Eqs. 8 and 9 are using data from signals originating at the point in space 1124. The example 1100 of spatial rolling encoding and decoding can be used to increase the frame rate. If, for example, N is the level of encoding, each ping 1116, 1118, and 1120 has two corresponding multi-mode signals with N=2. As detailed by Eqs. 6-9, data recovery here is four frames of data (two of RF₁ and two of RF₂). Each multi-mode signal 1118A and 1118B corresponds with two received data frames, giving the ping 1118 four recovered data frames with only two sent signals (the multi-modes 1118A and 1118B). In aspects, the frame rate for such a multi-mode rolling spatial encoding and decoding scheme with multi-modes of order N and outgoing signals of order M gives a frame rate F of:

$$F = M \cdot N \qquad Eq . 10$$

Though the discussion of the multi-mode rolling spatial encoding and decoding scheme of the example 1100 uses the example of two multi-modes per ping, with each multi-mode consisting of two mode types and using a Hadamard bipolar matrix operation for polarity and recombination, this should not be seen as limiting. Other orders for the pings and mode types can be used, as well as different encoding and decoding operators.

### Example User Interface

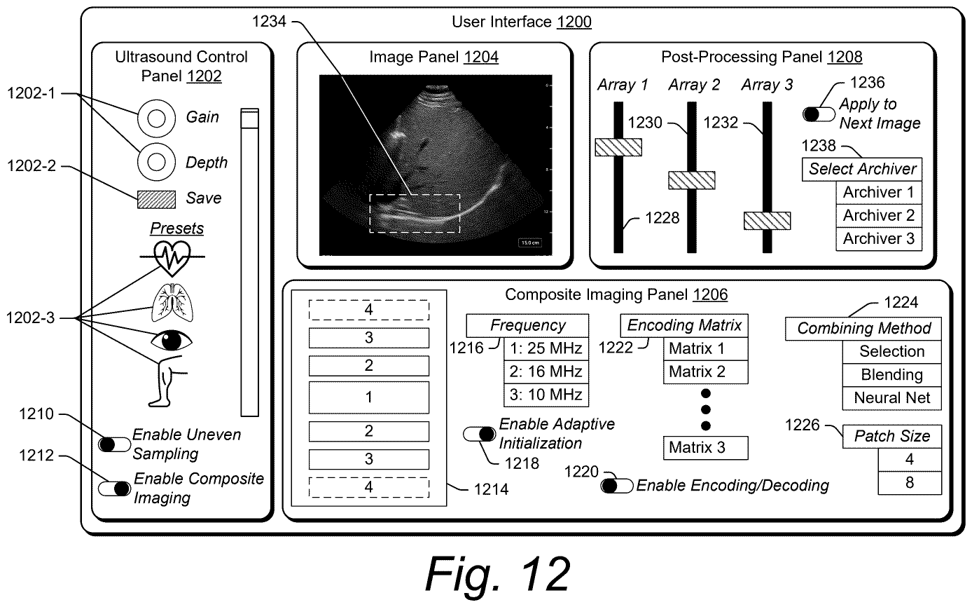

FIG. 12 illustrates an example user interface 1200 for multi-depth ultrasound imaging with a multi-frequency probe. The user interface 1200 can be displayed via an ultrasound machine (e.g., the ultrasound machine 102 of FIG. 1), a display device (e.g., the display 122 of FIG. 1), an ultrasound cart, and/or an ultrasound scanner (e.g., the ultrasound scanner 104 of FIG. 1). The user interface 1200 includes an ultrasound control panel 1202, an image panel 1204, a composite imaging panel 1206, and a post-processing panel 1208.

The ultrasound control panel 1202 includes any suitable controls and settings for controlling an ultrasound system, such as depth and gain adjustments 1202-1, and a button to store images and/or video clips 1202-2. The ultrasound control panel 1202 can also include icons to select examination presets 1202-3, such as a heart icon for a cardiac preset, a lung icon for a respiratory preset, an eye icon for an ocular preset, and a leg icon for a muscular-skeletal preset. The ultrasound control panel 1202 can also include options (not shown for clarity) to enable one or more neural networks for processing of an ultrasound image, such as an ultrasound image displayed in the image panel 1204. For instance, a cardiac neural network can be enabled to generate a value of ejection fraction, a free fluid network can be enabled to generate a segmentation of free fluid in an ultrasound image, and a pneumothorax (PTX) neural network can be enabled to generate a probability of a pneumothorax condition or collapsed lung.

The image panel 1204 can display any suitable ultrasound image, such as a B-mode image, an M-mode image, a Doppler image, a color flow image, etc. In an example, the image panel 1204 can display a composite image that is generated in accordance with the present disclosure. For example, a user can provide input to configure the system to generate a composite image by setting the controls and options in the composite imaging panel 1206 and enable imaging via an ultrasound scanner. The image panel 1204 can then display one or more composite images generated by the system that is configured according to the settings in the composite imaging panel 1206. Additionally or alternatively, the system can acquire data for generating a composite ultrasound image, such as data from a multi-array scanner (e.g., the multi-frequency probe 402 of FIG. 4). A user can then provide an input to the post-processing panel 1208 to cause the system to generate a new composite image based on user selections in the post-processing panel 1208. Then, the image panel 1204 can display the new composite image. In aspects, the image panel 1204 can also display a measurement of a parameter related to the anatomy, an annotation of the composite (or another) image, a classification of a portion of the anatomy, etc. In embodiments, the image panel 1204 can display an inference generated by a neural network, such as a segmentation of a blood vessel in a B-mode image. In aspects, the image panel 1204 can simultaneously display two or more images. For instance, the image panel 1204 can simultaneously display a first ultrasound image generated from data from a first transducer array (e.g., the first transducer array 404) that is configured to operate in a first imaging mode, and a second ultrasound image that is generated from data from a second transducer array (e.g., the second transducer array 408) that is configured to operate in a second imaging mode. As an example, the first ultrasound image can be a B-mode image, and the second ultrasound image can be a color Doppler image.

The ultrasound control panel 1202 also includes a user-selectable option 1210 (a switch, a slider, a button, etc.) to enable uneven sampling, as previously described. In FIG. 12, the user-selectable option 1210 is disabled. The ultrasound control panel 1202 also includes a user-selectable option 1212 (a switch, a slider, etc.) to enable composite imaging. In the example in FIG. 12, the user-selectable option 1212 is enabled. Responsive to enabling the user-selectable option 1212, the composite imaging panel 1206 can be displayed via the user interface 1200. The composite imaging panel 1206 can include any suitable data and user-selectable options for configuring an ultrasound system for ultrasound imaging in accordance with the present disclosure. In the example in FIG. 12, the composite imaging panel 1206 includes a visual representation 1214 of a multi-array transducer assembly. The visual representation 1214 depicts the multi-array transducer assembly with four arrays arranged in a component column. A first array includes a single component, denoted by the “1”, which represents a physical row of transducer elements on the multi-array transducer. A second array includes two components, denoted by the “2”, which represent two physical rows of transducer elements on the multi-array transducer that are adjacent to the center row. A third array includes two components, denoted by the “3”, which represent two physical rows of transducer elements on the multi-array transducer that are adjacent to the two rows of the second array, and a fourth array includes two components, denoted by the “4”, which represent two physical rows of transducer elements on the multi-array transducer that are adjacent to the two rows of the third array.

In the example in FIG. 12, a user has enabled the first array, the second array, and the third array (which are therefore illustrated with solid lines) and has disabled the fourth array (which is therefore illustrated with dashed lines). For instance, the user interface 1200 can be implemented via a touchscreen, and the user can provide an input (tap, double tap, mouse click, keyboard button actuation, etc.) on the arrays in the visual representation 1214 to successively enable and disable them. Responsive to enabling the first, second, and third arrays, the composite imaging panel 1206 displays a drop-down menu 1216 to set the frequencies of the selected arrays. In an example, the drop-down menu 1216 can provide options for setting transmission frequencies, reception frequencies, or both transmission and reception frequencies of each array. In the example in FIG. 12, the transmission and reception frequencies are the same for all components of an indicated array and are set to 25 MHz, 16 MHz, and 10 MHz for the first array, second array, and third array, respectively. It should be noted that the number of arrays and the available frequencies shown in the visual representation 1214 and the drop-down menu 1216 are illustrated in FIG. 12 as examples only. More or fewer options can be used, as well as more, fewer, and/or different frequencies.

The composite imaging panel 1206 also includes a user-selectable option 1218 to enable adaptive initialization of the system. As described above, when adaptive initialization is enabled, the system can generate one or more initialization frames that are not displayed in the user interface 1200. Rather, the system can determine any suitable imaging parameter for composite ultrasound imaging based on the initialization frames, such as a depth of pings, angles of ultrasound beams, regions for imaging, regions to avoid for imaging, regions with color to indicate fluid flow, SNR, contrast ratios, etc. Hence, the initialization frames can be based on one or more of the uneven sampling methods described herein, without the user-selectable option 1210 in the ultrasound control panel 1202 being explicitly enabled.

In aspects, the system determines the frequencies used by the arrays that are enabled in the visual representation 1214 based on the initialization frames. For instance, the system can use the frequencies set via the drop-down menu 1216 as baseline frequencies and then generate one or more initialization frames using the baseline frequencies and perturbations of the baseline frequencies. An example of a perturbation frequency is f+δf, where f is the baseline frequency set via the drop-down menu 1216 and δf is an offset term (which can be signed) to perturb the baseline frequency. The system can try any suitable number and combination of frequencies to generate any suitable number of initialization frames. The system can then select frequencies for the enabled arrays based on the initialization frames, such as according to a cost function of the initialization frames (e.g., the cost function of Eq. 1). For example, the cost function can generate an image score based on a combination of contrast ratio, SNR, etc. In an example, the system processes the initialization frames with a neural network, and the cost function can include an output of the neural network, such as an image score generated by the neural network, a segmentation, a quality of the segmentation, etc. In an example, the neural network generates a processor score that indicates an amount of computational resources needed to process the initialization frames, and the cost function can include the processor score generated by the neural network.

The composite imaging panel 1206 also includes a user-selectable option 1220 to enable encoding and decoding as previously described with respect to FIG. 11. The composite imaging panel 1206 also includes a drop-down menu 1222 to select a matrix for the encoding/decoding. The composite imaging panel 1206 also includes a drop-down menu 1224 to select a combining method for generating a composite image. Example combining methods include “selection,” “blending,” and “neural net.” For example, when the “selection” method is selected, the system can form a composite image with pixel values selected from images generated by different transducer arrays. In another example, when the “blending” method is selected, the system can form a composite image with pixel values formed by blending pixels from images generated by different transducer arrays. Blending can include adding, subtracting, averaging, performing a weighted average, etc. In another example, when the “neural net” method is selected, the system can form a composite image as the output of a neural network or other ML model that processes images generated by different transducer arrays. The composite imaging panel 1206 also includes a drop-down menu 1226 to select a patch size for a suitable combining method, such as “selection” or “blending”.

The user interface also includes the post-processing panel 1208, which can include any suitable option, control, etc. for processing a composite image after it has been generated by the system. In the example in FIG. 12, the post-processing panel 1208 includes sliders 1228, 1230, and 1232 for adjusting the data in the composite image from the first array, the second array, and the third array, respectively. In an example, the sliders 1228, 1230, and 1232 control depths of the composite image that correspond to the different arrays. In another example, the sliders 1228, 1230, and 1232 control relative numbers of pixels in the composite image from the three arrays. In still another example, the sliders 1228, 1230, and 1232 control relative weights of a pixel in the composite image from the three arrays. Although the post-processing panel 1208 is shown with the sliders 1228, 1230, and 1232 for the adjusting of the data in the composite image from the first array, the second array, and the third array, respectively, this is used as an example and should not be seen as limiting. For example, the sliders 1228, 1230, and 1232 could be knobs, discrete array values, etc.

In aspects, the image panel 1204 can display a composite image generated by the system, and the user can provide an input effective to draw an ROI 1234 on the composite image. The user can then provide an input effective to adjust relative contributions of the data from the three arrays within the ROI 1234 using the sliders 1228, 1230, and 1232. The user can then provide an input effective to draw another ROI on the composite image shown in the image panel 1204 (not shown for clarity) and then adjust the composite image in this new ROI using the sliders 1228, 1230, and 1232. The user can repeat this process any suitable number of times to generate a new composite image. The user can provide an input effective to enable the adjustments made via the sliders 1228, 1230, and 1232 to be applied to a new ultrasound image by enabling a switch 1236. For instance, the user can provide an input effective to adjust a current composite image displayed in the image panel 1204 via the drawing of one or more ROIs and adjustments of the sliders 1228, 1230, and 1232, which causes the system to generate new ultrasound data if the current ultrasound data is insufficient to render the new ultrasound image. The system can apply the adjustments in the ROIs made with the sliders 1228, 1230, and 1232 to the new ultrasound data to generate a new composite image. The post-processing panel 1208 also includes a drop-down menu 1238 to select an archiver (a medical archiver, DICOM archiver, VNA, etc.), and cause a composite image to be archived with the archiver.

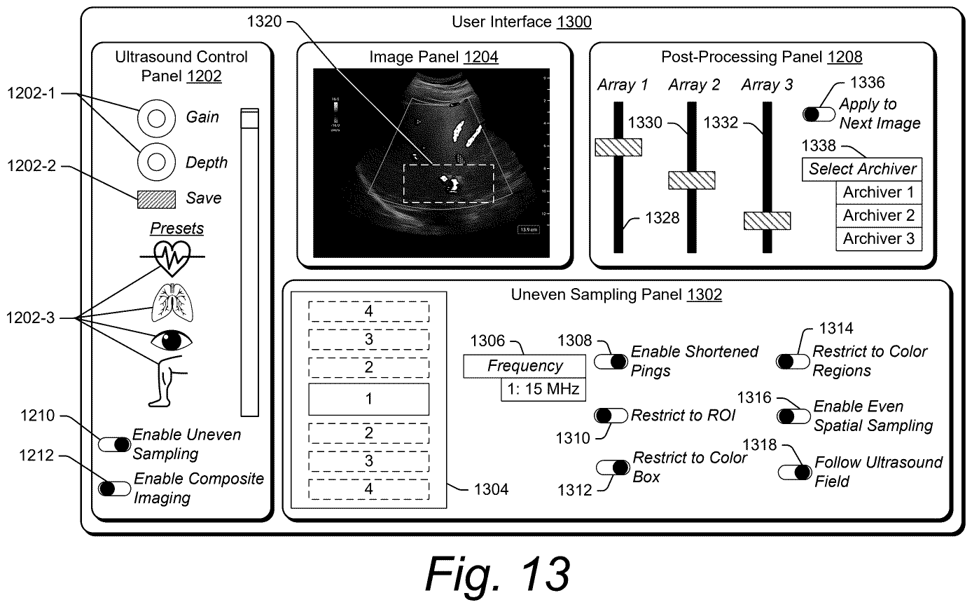

FIG. 13 illustrates an additional example embodiment 1300 for the example user interface 1200 of FIG. 12. The user interface 1300 is an example of the user interface 1200 in which the user-selectable option 1210 to enable uneven sampling in the ultrasound control panel 1202 is enabled. Responsive to the user-selectable option 1210 being enabled, the user interface 1300 can display an uneven sampling panel 1302.

The uneven sampling panel 1302 includes a visual representation 1304 of a multi-array transducer that contains the four arrays depicted in the visual representation 1214 in FIG. 12. However, unlike the example in FIG. 12, in FIG. 13, the user has provided input that has enabled the first array (having a center row of transducer elements), and disabled the second, third, and fourth arrays, as evidenced by the solid and dashed lines of the visual representation 1304. The user has also set the ultrasound frequency of the first array to 15 MHz via a drop-down menu 1306.

The uneven sampling panel 1302 also includes a user-selectable option 1308 to enable shortened pings, as previously described with respect to FIG. 6. In the example in FIG. 13, the user has enabled the user-selectable option 1308. The uneven sampling panel 1302 also includes a user-selectable option 1310 to restrict the ultrasound transmission to an ROI, which is disabled in this example. The uneven sampling panel 1302 also includes a user-selectable option 1312 to restrict the ultrasound transmission to a color region, which is enabled in this example. The image panel 1204 displays a color flow image having a color region 1320, and enabling the user-selectable option 1312 restricts the ultrasound transmission to the color region 1320. In an example, the system automatically determines the color region 1320, such as with a ML model. Additionally or alternatively, the user can provide an input effective to select (draw, select from computer-determined options, etc.) the color region 1320.

The uneven sampling panel 1302 also includes a user-selectable option 1314 to restrict the ultrasound transmission to color regions (e.g., the regions in the color region 1320 that have color to indicate fluid flow). The user-selectable option 1314 is disabled in the example in FIG. 13. The uneven sampling panel 1302 also includes a user-selectable option 1316 to enable even spatial sampling (e.g., enabling the far field to be sampled as frequently as the near field in a curved image). The user-selectable option 1316 is disabled in the example shown in FIG. 13. The uneven sampling panel 1302 also includes a user-selectable option 1318 to follow the ultrasound field, as previously described with respect to FIG. 10. The user-selectable option 1318 is enabled in the example of FIG. 13.

### Example Machine-Learning

As described, many of the features described herein can be implemented using a ML model (a neural network, a decision tree, etc.). For the purposes of this disclosure, a ML model is any model that accepts an input, analyzes and/or processes the input based on an algorithm derived via machine-learning training, and provides an output. A ML model can be conceptualized as a mathematical function of the following form:

$$f ( \hat{s} , θ ) = \hat{y} \qquad Eq . 11$$

In Eq. 11, the operator f represents the processing of the ML model based on an input and providing an output. The term ŝ represents a model input, such as ultrasound data from an ultrasound examination performed using a multi-frequency probe. The model analyzes/processes the input § using parameters θ to generate an output ŷ (the ROI 1234 of FIG. 12, one or more scanning parameters based on initialization frame inputs, etc.). Both ŝ and ŷ can be scalar values, matrices, vectors, or mathematical representations of phenomena such as categories, classifications, image characteristics, the images themselves, text, labels, or the like. The parameters θ can be any suitable mathematical operations, including but not limited to applications of weights and biases, filter coefficients, summations or other aggregations of data inputs, distribution parameters such as mean and variance in a Gaussian distribution, linear-algebra-based operators, or other parameters, including combinations of different parameters, suitable to map data to the desired output.

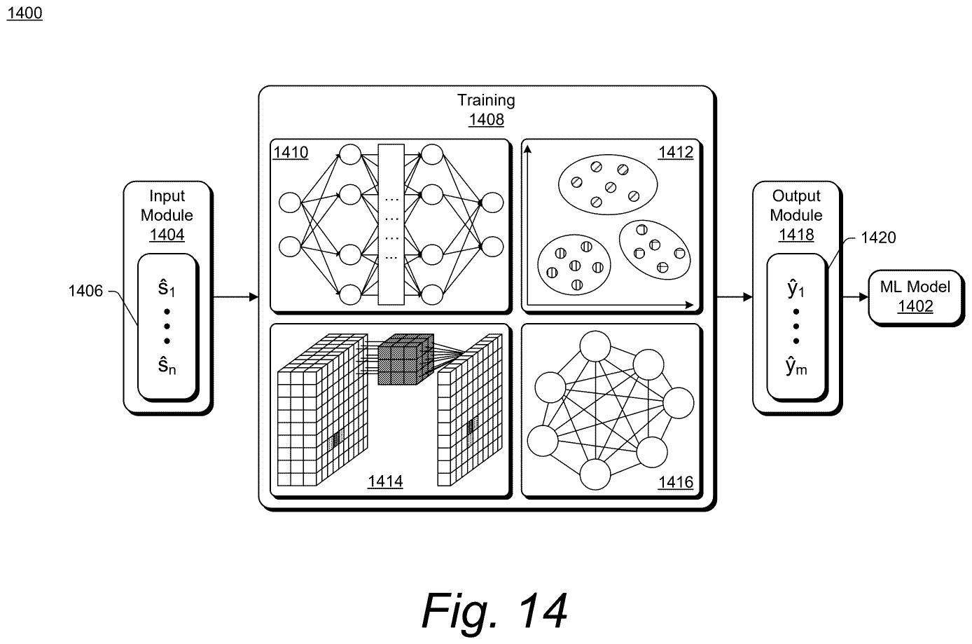

FIG. 14 represents an example machine-learning architecture 1400 used to train a ML model M 1402, which can be used to implement at least some of the techniques disclosed herein. An input module 1404 accepts an input ŝ 1406, which can be an array with members ŝ₁ through ŝₙ. The input ŝ 1406 is fed into a training module 1408, which processes the input ŝ 1406 based on the machine-learning architecture 1400. For example, if the machine-learning architecture 1400 uses a multilayer perceptron (MLP) model 1410, the training module 1408 applies weights and biases to the input ŝ 1406 through one or more layers of perceptrons, each perceptron performing a fit using its own weights and biases according to its given functional form. MLP weights and biases can be adjusted so that they are optimized against a least mean square, logcosh, or other optimization function (e.g., a loss function) known in the art. Although an MLP model 1410 is described here as an example, any suitable machine-learning technique can be employed, some examples of which include but are not limited to k-means clustering 1412, convolutional neural networks (CNN) 1414, a Boltzmann machine 1416, Gaussian mixture models (GMM), and long short-term memory (LSTM). The training module 1408 provides an input to an output module 1418. The output module 1418 analyzes the input from the training module 1408 and provides a prediction output in the form of ŷ 1420, which can be an array with members ŷ₁ through ŷₘ. The prediction output 1420 can represent a known correlation with the input ŷ 1406, such as, for example, anatomy information (e.g., characteristics of the patient anatomy 120).

In some examples, the input ŝ 1406 can be training input labeled with known output correlation values, and these known values can be used to optimize the output ŷ 1420 in training against the optimization/loss function. In other examples, the machine-learning architecture 1400 can categorize the output ŷ 1420 values without being given known correlation values to the input ŝ 1406. In some examples, the machine-learning architecture 1400 can be a combination of machine-learning architectures. By way of example, a first network can use the input ŝ 1406 and provide the prediction output ŷ 1420 as an input ŝ_{ML} to a second ML architecture, with the second ML architecture providing a final prediction output ŷ_{f}. In another example, one or more machine-learning architectures can be implemented at various points throughout the training module 1408.

In some ML models, all layers of the model are fully connected. For example, all perceptrons in an MLP model act on every member of ŝ. For an MLP model with a 100×100 pixel image as the input, each perceptron provides weights/biases for 10,000 inputs. With a large, densely layered model, this can result in slower processing and/or issues with vanishing and/or exploding gradients. A CNN, which may not be a fully connected model, can process the same image using 5×5 tiled regions, requiring only 25 perceptrons with shared weights, giving much greater efficiency than the fully connected MLP model.

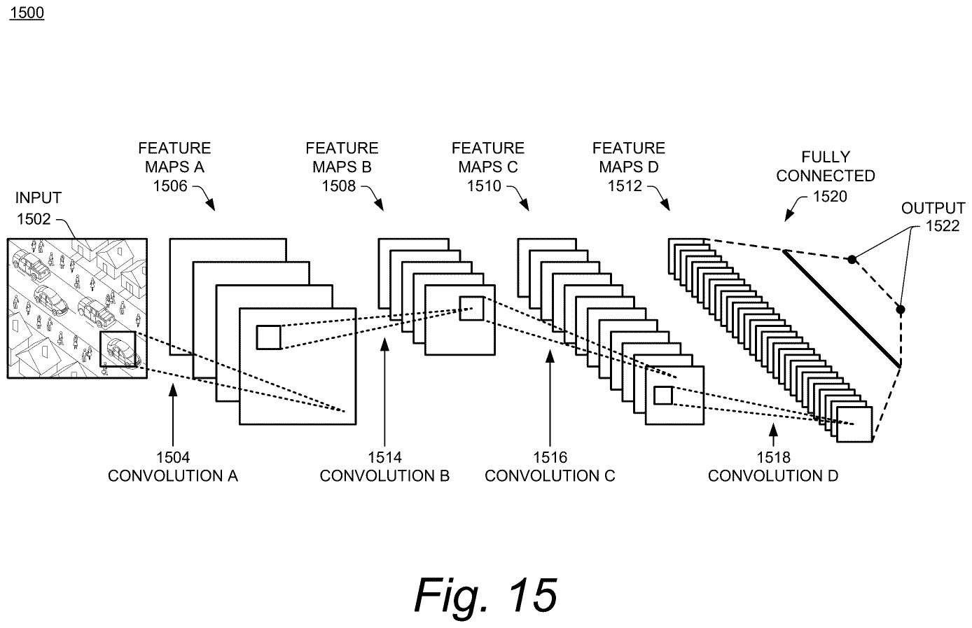

FIG. 15 represents an example ML model 1500 using a CNN to process an input image 1502, which includes representations of objects that can be identified via object recognition, such as organs, a fluid flow, a contrast or color element within an anatomy of a subject, a portion of the anatomy of the subject, etc. Although the illustrated input image 1502 includes people and cars as general objects, the input image 1502 can include the example image 800 of FIG. 8, as described above, having representations of color flow (the first color flow 804, the second color flow 806, etc.). Convolution A 1504 can be performed to create a first set of feature maps (e.g., feature maps A 1506). A feature map can be a mapping of aspects of the input image 1502 given by a filter element of the CNN. This process can be repeated using feature maps A 1506 to generate further feature maps B 1508, feature maps C 1510, and feature maps D 1512 using convolution B 1514, convolution C 1516, and convolution D 1518, respectively. In this example, feature maps D 1512 become an input for fully connected network layers 1520. In this way, the ML model can be trained to recognize certain elements of the image, such as people or cars, and provide an output 1522 that, for example, identifies the recognized elements.

Although the example of FIG. 15 shows a CNN as a part of a fully connected network, other architectures are possible and this example should not be seen as limiting. There can be more or fewer layers in the CNN. A CNN component for a model can be placed in a different order, or the model can contain additional components or models. There may be no fully connected components, such as a fully convolutional network. Additional aspects of the CNN, such as pooling, downsampling, upsampling, or other aspects known to people skilled in the art, can also be employed.

### Example Methods

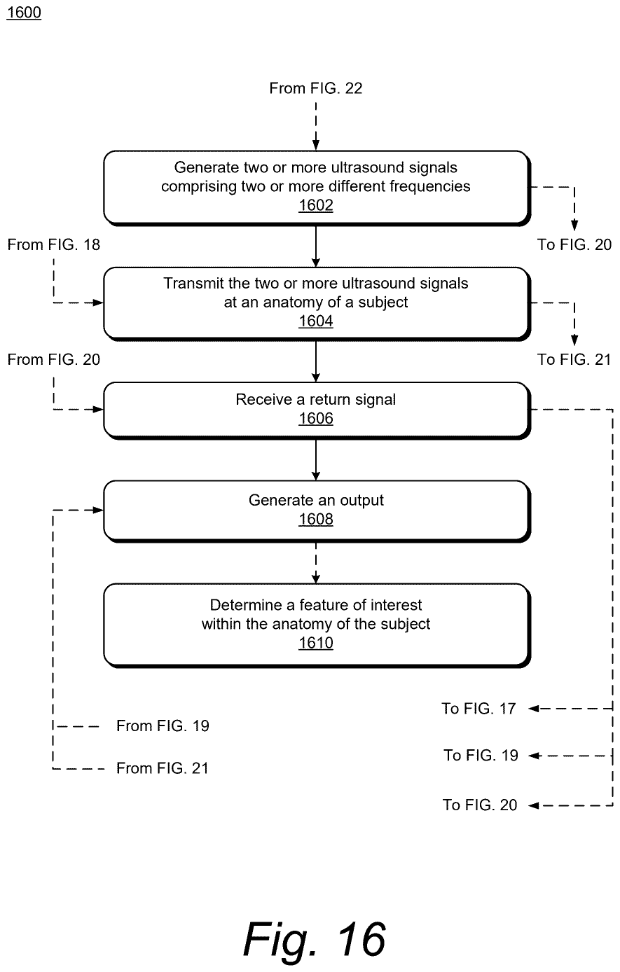

FIGS. 16-22 depict various methods 1600-2200, respectively, for multi-depth ultrasound imaging with a multi-frequency probe. The methods 1600-2200 are shown as sets of blocks that specify operations performed but are not necessarily limited to the order or combinations shown for performing the operations by the respective blocks. Further, any of one or more of the operations can be repeated, combined, reorganized, or linked to provide a wide array of additional and/or alternate methods. In portions of the following discussion, reference can be made to the example environment 100 of FIG. 1 or to entities or processes as detailed in FIGS. 2-15, reference to which is made for example only. The techniques are not limited to performance by one entity or multiple entities operating on one device. The methods 1600-2200 can be performed by an ultrasound machine, such as the ultrasound machine 102 of FIG. 1. Further, one or more operations from any of the methods 1600-2200 can be combined with one or more operations from any other of the methods 1600-2200 to generate additional and/or alternate methods, which are directly inferred from this disclosure.

FIG. 16 depicts the method 1600 for multi-depth ultrasound imaging with a multi-frequency probe. At 1602, two or more ultrasound signals are generated (the ultrasound beams 308, 310-1, 310-2, etc.). The two or more ultrasound signals are generated using a multi-array ultrasound scanner, such as the multi-frequency probe 302 of FIG. 3. In aspects, the two or more ultrasound signals are generated using two or more transducer arrays of the multi-array ultrasound scanner. The two or more ultrasound signals include two or more different frequencies. In some examples, each of the two or more ultrasound signals includes a different frequency than each of the other two or more ultrasound signals. In another example, at least two of the two or more ultrasound signals include a same frequency. In some examples, the two or more ultrasound signals include three or more ultrasound signals. In some examples, at least one of the frequencies of the two or more different frequencies represents a harmonic or a subharmonic of at least one other of the frequencies of the two or more different frequencies. The two or more ultrasound signals, in some examples, are configured to access two or more different depths of a subject.

At 1604, the two or more ultrasound signals are transmitted at the two or more different depths in an anatomy of the subject. The two or more ultrasound signals are transmitted by the two or more transducer arrays of the multi-array ultrasound scanner. The anatomy of the subject, in aspects, can include anatomies, organs, tissues, vessels, etc. In some examples, the transmission of the two or more ultrasound signals includes interleaving the two or more ultrasound signals by the two or more transducer arrays.

At 1606, a return signal is received. The return signal is based on the two or more ultrasound signals reflected from the two or more different depths in the anatomy of the subject. In some examples, the return signal is two or more return signals. In some examples, at least one of the two or more return signals is based on a different one of the two or more ultrasound signals reflected from the anatomy of the subject than at least one other of the two or more return signals.

At 1608, an output is generated (the example image 800, the example image 900, etc.). The output is generated based on the received return signal. The output is further based on at least two of the two or more different frequencies. In some examples, the output is an ultrasound image configured to be displayed on a display of an ultrasound machine (e.g., the display 122 of the ultrasound machine 102 of FIG. 1). The output, in some examples, is generated by a ML model, in accordance with this disclosure. The ML model can take one or more of the received return signal, the two or more different frequencies, and a configuration of the multi-array ultrasound scanner as inputs.

At 1610, in aspects, a feature of interest within the anatomy of the subject is determined. For example, the feature of interest can be a portion of the anatomy of the subject, such as an organ, a growth, etc. In some examples, the feature of interest is a fluid represented by a color flow (the first color flow 804, the second color flow 806, etc.). The feature of interest can be a pre-determined feature, a selection from a list of features, a determination from comparing ultrasound data to one or more threshold values, etc. According to some examples, the feature of interest is determined using a feature ML model. In aspects, the feature of interest is a portion of the anatomy of the user.

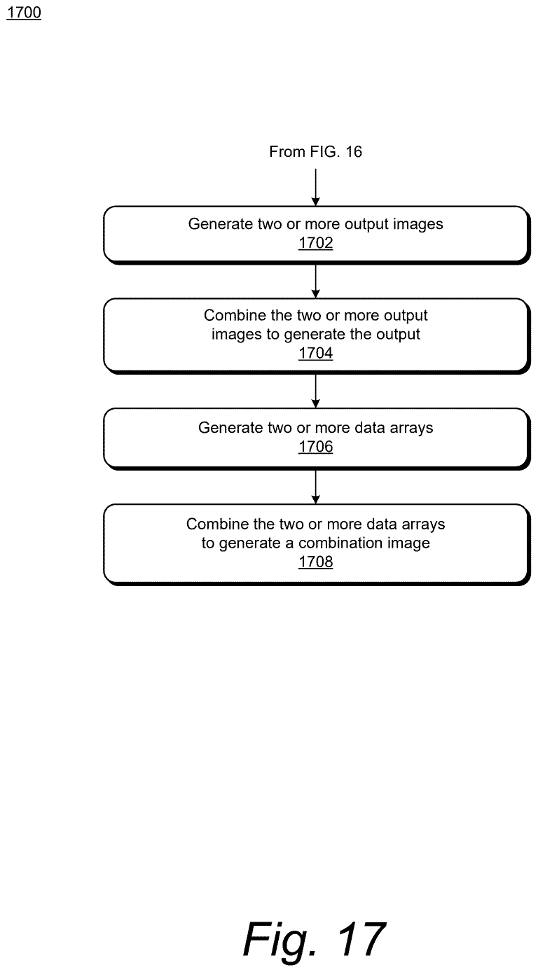

FIG. 17 depicts the method 1700 for multi-line nonlinear contrast imaging with ultrasound. At 1702, proceeding from 1606 in FIG. 16, two or more output images are generated. The two or more output images are generated based on the received return signal and at least two of the two or more different frequencies. At least one of the two or more output images is based on a different one of the two or more different frequencies than at least one other of the two or more output images.

At 1704, the two or more output images are combined to generate the output (e.g., at 1608 of FIG. 16). In aspects, the output is a combination image. In some examples, the combining of the two or more output images to generate the combination image is performed by a ML model. The two or more output images, in aspects, are provided to the ML model as an input. In some examples, the two or more output images are combined to generate the combination image based on a comparison of one or more pixels from a first image of the two or more output images with one or more pixels from a second image of the two or more output images, the first image being different than the second image. In some examples, the two or more output images are combined to generate the combination image based on a combination of one or more pixels from a first image of the two or more output images with one or more pixels from a second image of the two or more output images, the first image being different than the second image. In some examples, the one or more pixels from the first image of the two or more output images are combined with the one or more pixels from the second image of the two or more output images based on a summation, an average, a weighted average, a cost-function-based combination, or a machine-learning-based combination of the one or more pixels from the first image of the two or more output images and the one or more pixels from the second image of the two or more output images.

At 1706, two or more data arrays are generated. The two or more data arrays are each based on a different one of the two or more output images. In aspects, there is one data array of the two or more data arrays for each of the two or more output images, giving a one-to-one correspondence between the two or more data arrays and the two or more images.

At 1708, the two or more data arrays are combined to generate the combination image. In some examples, the two or more data arrays are combined based on a summation, an average, a weighted average, a cost-function-based combination, or a machine-learning-based combination of the data arrays. In some examples, the two or more data arrays are combined based on a comparison between the two or more data arrays, such as a pixel-by-pixel comparison. In some examples, the two or more data arrays are combined based on a subtraction.

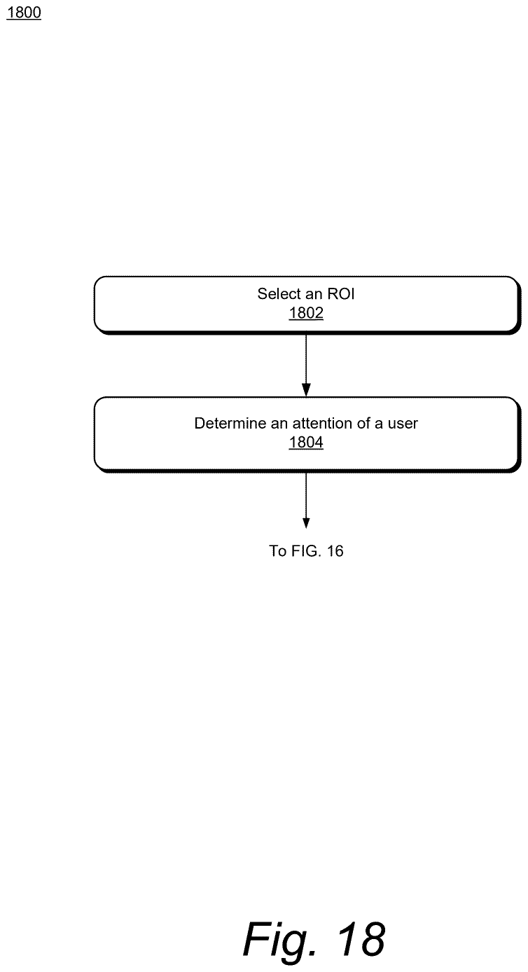

FIG. 18 depicts the method 1800 for multi-line nonlinear contrast imaging with ultrasound. At 1802, an ROI is selected. The ROI (the color region 802, the ROI 1234, etc.) is part of an anatomy of a subject under observation during an ultrasound procedure using a multi-frequency probe (e.g., the scanner 104 of FIG. 1). The ROI, in aspects, is within an available field of view of the multi-frequency probe. In some examples, the selection of the ROI is performed by a ML model. In other examples, the ROI is selected by a user of an ultrasound device (e.g., the ultrasound machine 102 of FIG. 1), the ultrasound device including the multi-frequency probe.

At 1804, an attention of the user is determined. In aspects, the attention of the user is a direction of focus or a point of interest of the user and can be based on an object being interacted with by the user. In aspects, the ROI is determined based on the attention of the user. In some examples, the determination of the ROI based on the attention of the user is performed by the ML model. In some examples, the attention of the user is determined based on a direction of a gaze of the user. In another example, the attention of the user is determined based on a UI element the user is interacting with (the image panel 1204, the post-processing panel 1208, etc.). According to some examples, the method 1800 proceeds to 1604 in FIG. 6.

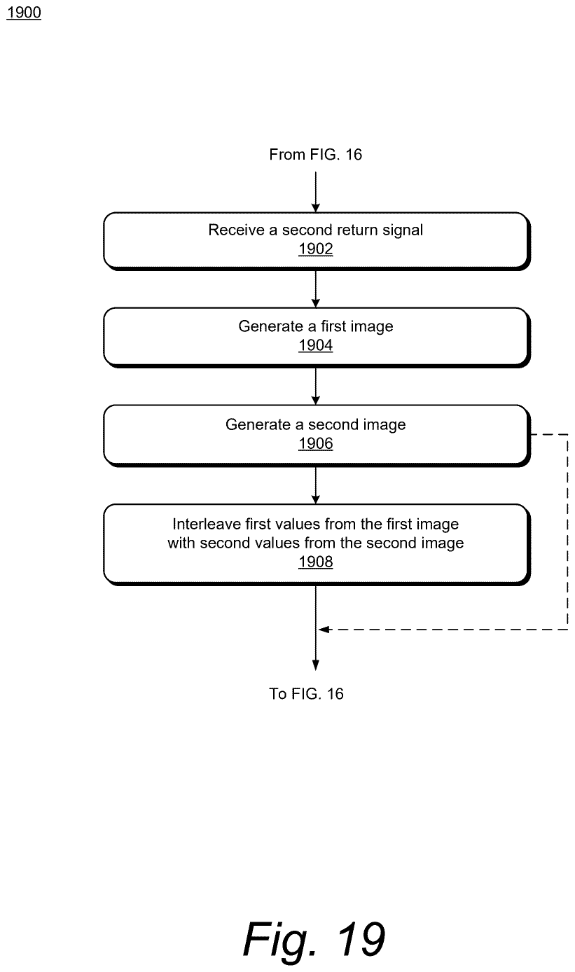

FIG. 19 depicts the method 1900 for multi-line nonlinear contrast imaging with ultrasound. In the method 1900, the return signal at 1606 of the method 1600 represents a first return signal based on at least a first frequency of the two or more different frequencies. At 1902, a second return signal is received. The second return signal is based on the two or more ultrasound signals reflected from the anatomy of the subject and at least a second frequency of the two or more different frequencies, the second frequency different than the first frequency. In some examples, the first return signal is based on the first frequency and no other frequencies of the two or more different frequencies and the second return signal is based on the second frequency and no other frequencies of the two or more different frequencies.

At 1904, a first image is generated. The first image is based on the received first return signal and the first frequency. At 1906, a second image is generated. The second image is based on the received second return signal and the second frequency. In some aspects, the method 1900 proceeds to 1608 of FIG. 16 such that the generation of the output at 1608 of FIG. 16 is based on the first image and the second image.

At 1908, first values from the first image are interleaved with second values from the second image. The interleaving can, in aspects, be performed by one or more processors of the ultrasound machine (e.g., the one or more processors 106 of FIG. 1). In aspects, method 1900 proceeds to 1608 of FIG. 16 such that the generation of the output at 1608 of FIG. 16 is based on the interleaving of the first values from the first image with the second values from the second image.

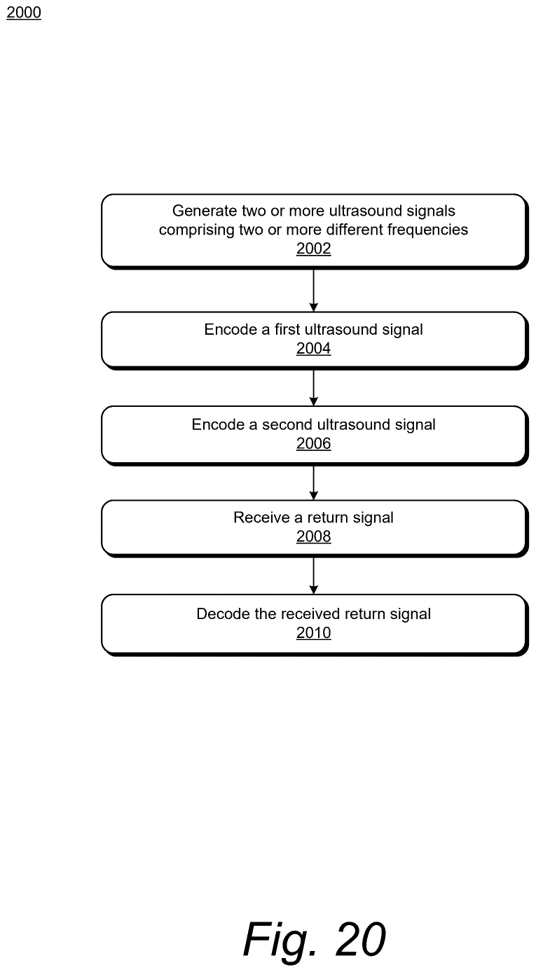

FIG. 20 depicts the method 2000 for multi-line nonlinear contrast imaging with ultrasound. At 2002, two or more ultrasound signals are generated (the ultrasound beams 308, 310-1, 310-2, etc.). The two or more ultrasound signals are generated using a multi-array ultrasound scanner, such as the multi-frequency probe 302 of FIG. 3. In aspects, the two or more ultrasound signals are generated using two or more transducer arrays of the multi-array ultrasound scanner. The two or more ultrasound signals include two or more different frequencies. In some examples, each of the two or more ultrasound signals includes a different frequency than each of the other two or more ultrasound signals. In another example, at least two of the two or more ultrasound signals include a same frequency. In some examples, the two or more ultrasound signals include three or more ultrasound signals. In some examples, at least one of the frequencies of the two or more different frequencies represents a harmonic or a subharmonic of at least one other of the frequencies of the two or more different frequencies.

The two or more ultrasound signals include a first ultrasound signal of the two or more ultrasound signals generated using a first frequency of the two or more different frequencies. The two or more ultrasound signals further include a second ultrasound signal of the two or more ultrasound signals generated using a second frequency of the two or more different frequencies. The second frequency is different than the first frequency. At 2004, the first ultrasound signal is encoded. In some examples, the first ultrasound signal is encoded by one or more processors of the ultrasound machine (e.g., the one or more processors 106 of FIG. 1). In other examples, the first ultrasound signal is encoded by the two or more transducer arrays (the arrays 304, 306-1, 306-2, 404, etc.).

At 2006, the second ultrasound signal is encoded. In some examples, the second ultrasound signal is encoded by the one or more processors. In other examples, the second ultrasound signal is encoded by the two or more transducer arrays. In some examples, the second ultrasound signal is encoded using one or more phase-inverted ultrasound signals of the first ultrasound signal.

At 2008, a return signal is received. The return signal is based on the two or more ultrasound signals reflected from the anatomy of the subject. In some examples, the return signal is two or more return signals. In some examples, at least one of the two or more return signals is based on a different one of the two or more ultrasound signals reflected from the anatomy of the subject than at least one other of the two or more return signals.

At 2010, the received return signal is decoded. The received return signal is decoded, in aspects, based on the encoded first ultrasound signal and the encoded second ultrasound signal. In some examples, the received return signal is decoded by a decoding ML model. In some examples, the received return signal includes a first return signal based on the first ultrasound signal reflected from the anatomy of the subject and a second return signal based on the second ultrasound signal reflected from the anatomy of the subject. In some examples, the received return signal is decoded based on a combination of the first return signal and the second return signal. The first return signal, in some examples, is combined with the second return signal based on a summation, an average, a weighted average, a cost-function-based combination, or a machine-learning-based combination.

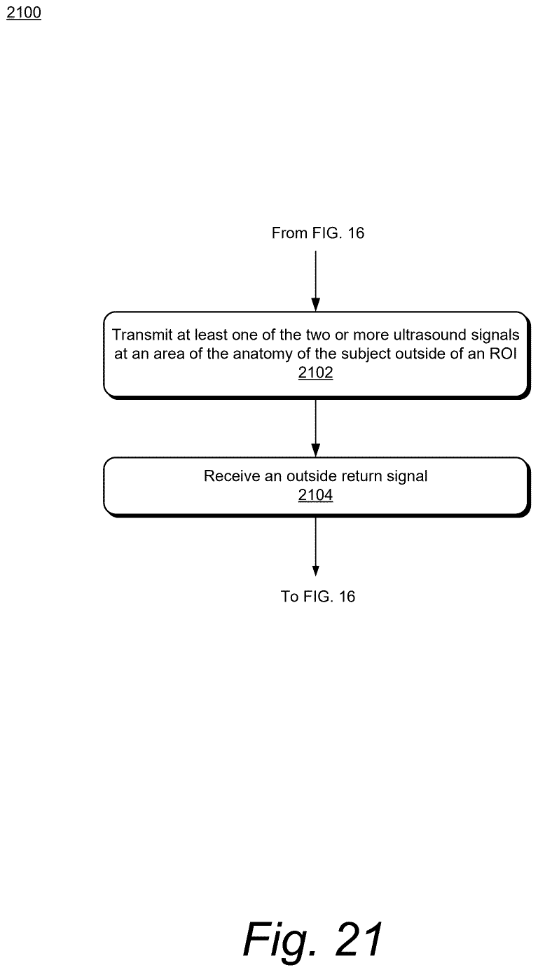

FIG. 21 depicts the method 2100 for multi-line nonlinear contrast imaging with ultrasound and proceeds from the operation 1604 of FIG. 16. In aspects, the transmission of the two or more ultrasound signals includes one or more asymmetric transmission characteristics. In some examples, the one or more asymmetric transmission characteristics for the two or more ultrasound signals include one or more of a depth, a time, a rate, or an angle of probing of the anatomy of the subject. In some examples, the one or more asymmetric characteristics are based on a presence of a contrast element within the anatomy of the subject. At 2102, at least one of the two or more ultrasound signals is transmitted at an area of the anatomy of the subject outside of an ROI. For example, the two or more ultrasound signals are transmitted at the anatomy in areas corresponding to regions of the image in the image panel 1204 outside of the ROI 1234 in FIG. 12.

At 2104, an outside return signal is received. The outside return signal, in aspects, is based on the at least one of the two or more ultrasound signals transmitted at the area of the anatomy of the subject outside of the ROI. The output (e.g., at 1608 of FIG. 16), in aspects, is further generated based on the outside return signal and further includes information corresponding to the at least one of the two or more ultrasound signals reflected from the area of the subject outside of the ROI. In some examples, the one or more asymmetric characteristics are based on a shape of an ultrasound field and the ultrasound field includes the two or more transmitted ultrasound signals. In some examples, the one or more asymmetric characteristics are determined using a determination ML model.

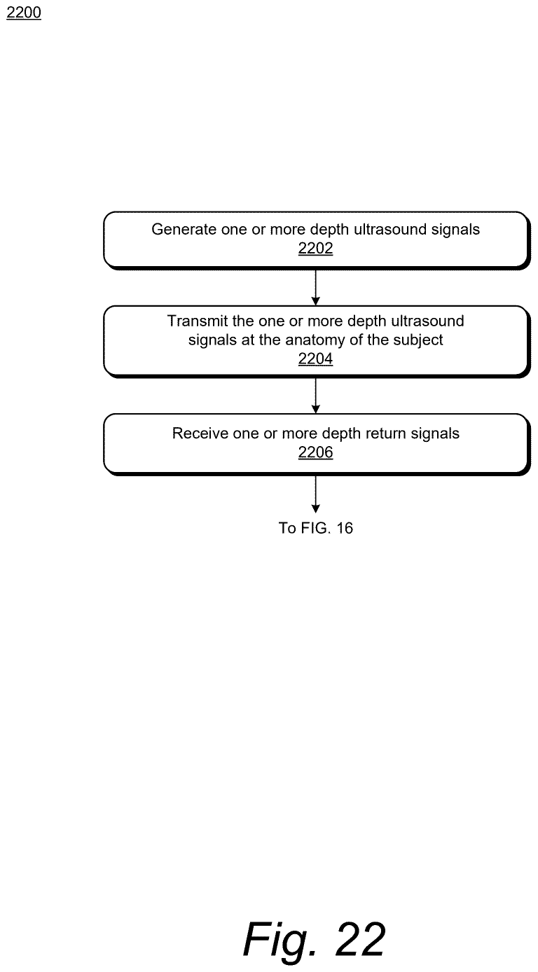

FIG. 22 depicts the method 2200 for multi-line nonlinear contrast imaging with ultrasound. In aspects, the method 2200 includes the method 1600 of FIG. 16. At 2202, one or more depth ultrasound signals are generated. The one or more depth ultrasound signals are configured to determine a depth of penetration into the anatomy of the subject.

At 2204, the one or more depth ultrasound signals are transmitted at the anatomy of the subject. At 2206, one or more depth return signals are received. The one or more depth return signals, in aspects, are based on the one or more depth ultrasound signals reflected from the anatomy of the subject. The method 2200 then proceeds to 1602 in FIG. 16 where the generation of the two or more ultrasound signals is based on the one or more depth return signals.

### Additional Examples

The following are additional examples of the described devices and methods for multi-depth ultrasound imaging with a multi-frequency probe.

Example 1: An ultrasound device including a multi-array ultrasound scanner including two or more transducer arrays. The multi-array ultrasound scanner is configured to generate, using the two or more transducer arrays, two or more ultrasound signals including two or more different frequencies. The multi-array ultrasound scanner is further configured to transmit the two or more ultrasound signals at an anatomy of a subject and receive a return signal based on the two or more ultrasound signals reflected from the anatomy of the subject. The ultrasound device further includes one or more processors and a memory, the memory storing instructions that, when executed by the one or more processors, cause the one or more processors to generate an output. The output is based on the received return signal and at least two of the two or more different frequencies associated with the two or more ultrasound signals reflected from the anatomy of the subject.

Example 2: The ultrasound device of example 1, where the return signal is two or more return signals. At least one of the two or more return signals is based on a different one of the two or more ultrasound signals reflected from the anatomy of the subject than at least one other of the two or more return signals.

Example 3: The ultrasound device of example 1, where the output is a combination image and the instructions further cause the one or more processors to generate two or more output images. At least one of the two or more output images generated is based on a different one of the two or more different frequencies than at least one other of the two or more output images. The instructions further cause the one or more processors to combine the two or more output images to generate the combination image.

Example 4: The ultrasound device of example 3, where the two or more output images are combined to generate the combination image by a machine-learned model. At least the two or more output images are provided as an input to the machine-learned model.

Example 5: The ultrasound device of example 3, where the two or more output images are combined to generate the combination image based on a comparison of one or more pixels from a first image of the two or more output images with one or more pixels from a second image of the two or more output images, the first image being different than the second image.

Example 6: The ultrasound device of example 5, where the two or more output images are combined to generate the combination image based on a combination of one or more pixels from the first image of the two or more output images with one or more pixels from a second image of the two or more output images, the first image being different than the second image.

Example 7: The ultrasound device of example 6, where the one or more pixels from the first image of the two or more output images are combined with the one or more pixels from the second image of the two or more output images based on a summation, an average, a weighted average, a cost-function-based combination, or a machine-learning-based combination of the one or more pixels from the first image of the two or more output images and the one or more pixels from the second image of the two or more output images.

Example 8: The ultrasound device of example 3, where the instructions further cause the one or more processors to generate two or more data arrays. Each of the two or more data arrays is based on a different one of the two or more output images, where the two or more output images are combined to generate the combined image based on the two or more data arrays.

Example 9: The ultrasound device of example 8, where the two or more output images are combined based on a summation, an average, a weighted average, a cost-function-based combination, or a machine-learning-based combination of the data arrays.

Example 10: The ultrasound device of example 1, where the output is generated by the one or more processors using a machine-learned model, where at least the received return signal is provided as an input to the machine-learned model.

Example 11: The ultrasound device of example 1, where the instructions further cause the one or more processors to select a region of interest (ROI) from an available field of view of the multi-array ultrasound scanner.

Example 12: The ultrasound device of example 11, where the selection of the ROI is performed by a machine-learned model.

Example 13: The ultrasound device of example 1, where the instructions further cause the one or more processors to determine, based on the generated output, a feature of interest within the anatomy of the subject.

Example 14: The ultrasound device of example 13, where the feature of interest is determined using a machine-learned model.

Example 15: The ultrasound device of example 13, where the feature of interest is a portion of the anatomy of the subject.

Example 16: The ultrasound device of example 1, where the return signal is a first return signal based on at least a first frequency of the two or more different frequencies associated with the two or more ultrasound signals reflected from the anatomy of the subject. The instructions further cause the one or more processors to receive a second return signal, the second return signal based on the two or more ultrasound signals reflected from the anatomy of the subject and at least a second frequency of the two or more different frequencies associated with the two or more ultrasound signals reflected from the anatomy of the subject. The second frequency is different than the first frequency. The instructions further cause the one or more processors to generate, based on the received first return signal and the first frequency, a first image and generate, based on the received second return signal and the second frequency, a second image, where the output is further based on the first image and the second image.

Example 17: The ultrasound device of example 16, where the instructions further cause the one or more processors to interleave first values from the first image with second values from the second image. The output is further based on the interleaving of the first values from the first image with the second values from the second image.

Example 18: The ultrasound device of example 1, where the two or more ultrasound signals include three or more ultrasound signals.

Example 19: The ultrasound device of example 1, where the multi-array ultrasound scanner is further configured to interleave the two or more ultrasound signals by the two or more transducer arrays.

Example 20: The ultrasound device of example 1, where at least one of the frequencies of the two or more ultrasound signals represents a harmonic or a subharmonic of at least one other of the frequencies of the two or more ultrasound signals.

Example 21: The ultrasound device of example 1, where the two or more ultrasound signals include a first ultrasound signal of the two or more ultrasound signals generated using a first frequency and a second ultrasound signal of the two or more ultrasound signals generated using a second frequency, where the second frequency is different than the first frequency.

Example 22: The ultrasound device of example 21, where the multi-array ultrasound scanner is further configured to encode the first ultrasound signal and encode the second ultrasound signal.

Example 23: The ultrasound device of example 22, where the second ultrasound signal is encoded using one or more phase-inverted ultrasound signals of the first ultrasound signal.

Example 24: The ultrasound device of example 22, where the instructions further cause the one or more processors to decode the received return signal based on the encoded first ultrasound signal and the encoded second ultrasound signal.

Example 25: The ultrasound device of example 24, where the received return signal is decoded using a machine-learned model.

Example 26: The ultrasound device of example 24, where the received return signal includes a first return signal based on the first ultrasound signal reflected from the anatomy of the subject and a second return signal based on the second ultrasound signal reflected from the anatomy of the subject. The received return signal is decoded by combining the first return signal and the second return signal.

Example 27: The ultrasound device of example 26, where the instructions further cause the one or more processors to combine the first return signal and the second return signal based on a summation, an average, a weighted average, a cost-function-based combination, or a machine-learning-based combination.

Example 28: A user interface (UI) for an ultrasound device, the user interface configured to register one or more user inputs. The one or more user inputs cause one or more processors to generate, by two or more transducer arrays of a multi-array ultrasound scanner, two or more ultrasound signals. Each of the two or more ultrasound signals is generated using at least two different frequencies. The one or more user inputs further cause the one or more processors to transmit, using the two or more transducer arrays, the two or more ultrasound signals at an anatomy of a subject and receive, using the two or more transducer arrays, a return signal based on the two or more ultrasound signals reflected from the anatomy of the subject. The one or more user inputs further cause the one or more processors to generate, based on the received return signal, an output, the output based on the received return signal and at least two of the different frequencies associated with the two or more ultrasound signals reflected from the anatomy of the subject.

Example 29: The UI of example 28, where the return signal is two or more return signals, at least one of the two or more return signals based on a different one of the two or more ultrasound signals reflected from the anatomy of the subject than at least one other of the two or more return signals.

Example 30: The UI of example 29, where the output is a combination image and the one or more user inputs further cause the one or more processors to generate two or more output images, at least one of the two or more output images generated based on a different one of the two or more different frequencies than at least one other of the two or more output images; and combine the two or more output images to generate the combination image.

Example 31: The UI of example 30, where the two or more output images are combined to generate the combination image by a machine-learned model, where at least the two or more output images are provided as an input to the machine-learned model.

Example 32: The UI of example 30, where the two or more output images are combined to generate the combination image based on a comparison of one or more pixels from a first image of the two or more output images with one or more pixels from a second image of the two or more output images, the first image being different than the second image.

Example 33: The UI of example 32, where the two or more output images are combined to generate the combination image based on a combination of one or more pixels from the first image of the two or more output images with one or more pixels from a second image of the two or more output images, the first image being different than the second image.

Example 34: The UI of example 33, where the one or more pixels from the first image of the two or more output images are combined with the one or more pixels from the second image of the two or more output images based on a summation, an average, a weighted average, a cost-function-based combination, or a machine-learning-based combination of the one or more pixels from the first image of the two or more output images and the one or more pixels from the second image of the two or more output images.

Example 35: The UI of example 30, where the one or more user inputs further cause the one or more processors to generate two or more data arrays, each of the two or more data arrays based on a different one of the two or more output images, where the two or more output images are combined to generate the combined image based on the two or more data arrays.

Example 36: The UI of example 35, where the two or more output images are combined based on a summation, an average, a weighted average, a cost-function-based combination, or a machine-learning-based combination of the data arrays.

Example 37: The UI of example 28, where the output is generated by the one or more processors using a machine-learned model, where at least the received return signal is provided as an input to the machine-learned model.

Example 38: The UI of example 28, where the one or more user inputs further cause the one or more processors to select a region of interest (ROI) within the anatomy of the subject from an available field of view of the multi-array ultrasound scanner.

Example 39: The UI of example 38, where the selection of the ROI is performed by a machine-learned model.

Example 40: The UI of example 38, where the one or more user inputs further cause the one or more processors to determine an attention of an operator of the ultrasound device and the ROI is selected based on the user attention.

Example 41: The UI of example 40, where the attention of the operator of the ultrasound device is determined based on a direction of a gaze of the operator of the ultrasound device.

Example 42: The UI of example 28, where the one or more user inputs further cause the one or more processors to determine, based on the generated output, a feature of interest within the anatomy of the subject.

Example 43: The UI of example 42, where the feature of interest is determined using a machine-learned model.

Example 44: The UI of example 42, where the feature of interest is a portion of the anatomy of the subject.

Example 45: The UI of example 28, where the return signal is a first return signal based on at least a first frequency of the two or more different frequencies associated with the two or more ultrasound signals reflected from the anatomy of the subject, where the one or more user inputs further cause the one or more processors to receive a second return signal, the second return signal based on the two or more ultrasound signals reflected from the anatomy of the subject and at least a second frequency of the two or more different frequencies associated with the two or more ultrasound signals reflected from the anatomy of the subject, the second frequency being different than the first frequency. The one or more user inputs further cause the one or more processors to generate, based on the received first return signal and the first frequency, a first image and generate, based on the received second return signal and the second frequency, a second image, where the output is further based on the first image and the second image.

Example 46: The UI of example 45, where the one or more user inputs further cause the one or processors to interleave first values from the first image with second values from the second image and the output is further based on the interleaving of the first values from the first image with the second values from the second image.

Example 47: The UI of example 28, where the two or more ultrasound signals include three or more ultrasound signals.

Example 48: The UI of example 28, where the transmission of the two or more ultrasound signals includes interleaving the two or more ultrasound signals by the two or more transducer arrays.

Example 49: The UI of example 28, where at least one of the frequencies of the two or more ultrasound signals represents a harmonic or a subharmonic of at least one other of the frequencies of the two or more ultrasound signals.

Example 50: The UI of example 28, where the two or more ultrasound signals include a first ultrasound signal of the two or more ultrasound signals generated using a first frequency and a second ultrasound signal of the two or more ultrasound signals generated using a second frequency, where the second frequency is different than the first frequency.

Example 51: The UI of example 50, where the one or more user inputs further cause the one or more processors to encode the first ultrasound signal and encode the second ultrasound signal.

Example 52: The UI of example 51, where the second ultrasound signal is encoded using one or more phase-inverted ultrasound signals of the first ultrasound signal.

Example 53: The UI of example 51, where the one or more user inputs further cause the one or more processors to decode the received return signal based on the encoded first ultrasound signal and the encoded second ultrasound signal.

Example 54: The UI of example 53, where the received return signal is decoded using a machine-learned model.

Example 55: The UI of example 53, where the received return signal includes: a first return signal based on the first ultrasound signal reflected from the anatomy of the subject and a second return signal based on the second ultrasound signal reflected from the anatomy of the subject. The received return signal is decoded based on a combination of the first return signal and the second return signal.

Example 56: The UI of example 55, where the first return signal is combined with the second return signal based on a summation, an average, a weighted average, a cost-function-based combination, or a machine-learning-based combination.

Example 57: A method for nonlinear contrast imaging with ultrasound, the method including generating, using two or more transducer arrays of a multi-array ultrasound scanner, two or more ultrasound signals comprising two or more different frequencies. The method further includes transmitting, using the two or more transducer arrays, the two or more ultrasound signals at an anatomy of a subject and receiving, using the two or more transducer arrays, a return signal based on the two or more ultrasound signals reflected from the anatomy of the subject. The method further includes generating, by one or more processors, an output. The output is based on the received return signal and the two or more different frequencies.

Example 58: The method of example 57, where the return signal is two or more return signals, at least one of the two or more return signals based on a different one of the two or more ultrasound signals reflected from the anatomy of the subject than at least one other of the two or more return signals.

Example 59: The method of example 58, where the output is a combination image and the method further includes generating two or more output images, at least one of the two or more output images generated based on a different one of the two or more different frequencies than at least one other of the two or more output images, and combining the two or more output images to generate the combination image.

Example 60: The method of example 59, where the two or more output images are combined to generate the combination image by a machine-learned model, where at least the two or more output images are provided as an input to the machine-learned model.

Example 61: The method of example 59, where the two or more output images are combined to generate the combination image based on a comparison of one or more pixels from a first image of the two or more output images with one or more pixels from a second image of the two or more output images, the first image being different than the second image.

Example 62: The method of example 61, where the two or more output images are combined to generate the combination image based on a combination of one or more pixels from the first image of the two or more output images with one or more pixels from a second image of the two or more output images, the first image being different than the second image.

Example 63: The method of example 62, where the one or more pixels from the first image of the two or more output images are combined with the one or more pixels from the second image of the two or more output images based on a summation, an average, a weighted average, a cost-function-based combination, or a machine-learning-based combination of the one or more pixels from the first image of the two or more output images and the one or more pixels from the second image of the two or more output images.

Example 64: The method of example 59, further including generating two or more data arrays, each of the two or more data arrays based on a different one of the two or more output images, where the two or more output images are combined to generate the combined image based on the two or more data arrays.

Example 65: The method of example 64, where the two or more output images are combined based on a summation, an average, a weighted average, a cost-function-based combination, or a machine-learning-based combination of the data arrays.

Example 66: The method of example 57, where the output is generated by the one or more processors using a machine-learned model, where at least the received return signal is provided as an input to the machine-learned model.

Example 67: The method of example 57, further including selecting, by the one or more processors, a region of interest (ROI) within the anatomy of the subject from an available field of view of the multi-array ultrasound scanner.

Example 68: The method of example 67, where the selection of the ROI is performed by a machine-learned model.

Example 69: The method of example 67, further including determining, by the one or more processors, an attention of an operator of the ultrasound device, where the ROI is selected based on the user attention.

Example 70: The method of example 69, where the attention of the operator of the ultrasound device is determined based on a direction of a gaze of the operator of the ultrasound device.

Example 71: The method of example 57, further including determining, by the one or more processors and based on the generated output, a feature of interest within the anatomy of the subject.

Example 72: The method of example 71, where the feature of interest is determined using a machine-learned model.

Example 73: The method of example 71, where the feature of interest is a portion of the anatomy of the subject.

Example 74: The method of example 57, where the return signal is a first return signal based on at least a first frequency of the two or more different frequencies associated with the two or more ultrasound signals reflected from the anatomy of the subject, the method further including receiving a second return signal, the second return signal based on the two or more ultrasound signals reflected from the anatomy of the subject; and at least a second frequency of the two or more different frequencies associated with the two or more ultrasound signals reflected from the anatomy of the subject, the second frequency being different than the first frequency; generating, by the one or more processors and based on the received first return signal and the first frequency, a first image; and generating, by the one or more processors and based on the received second return signal and the second frequency, a second image, where the output is further based on the first image and the second image.

Example 75: The method of example 74, further including interleaving first values from the first image with second values from the second image, where the output is further based on the interleaving of the first values from the first image with the second values from the second image.

Example 76: The method of example 57, where the two or more ultrasound signals include three or more ultrasound signals.

Example 77: The method of example 57, where the transmission of the two or more ultrasound signals includes interleaving the two or more ultrasound signals by the two or more transducer arrays.

Example 78: The method of example 57, where at least one of the frequencies of the two or more ultrasound signals represents a harmonic or a subharmonic of at least one other of the frequencies of the two or more ultrasound signals.

Example 79: The method of example 57, where the two or more ultrasound signals include: a first ultrasound signal of the two or more ultrasound signals generated using a first frequency and a second ultrasound signal of the two or more ultrasound signals generated using a second frequency, where the second frequency is different than the first frequency.

Example 80: The method of example 79, further including encoding, by the one or more processors or the two or more transducer arrays, the first ultrasound signal and encoding, by the one or more processors or the two or more transducer arrays, the second ultrasound signal.

Example 81: The method of example 80, where the second ultrasound signal is encoded using one or more phase-inverted ultrasound signals of the first ultrasound signal.

Example 82: The method of example 80, further including decoding, by the one or more processors, the received return signal based on the encoded first ultrasound signal and the encoded second ultrasound signal.

Example 83: The method of example 82, where the received return signal is decoded using a machine-learned model.

Example 84: The method of example 82, where the received return signal includes a first return signal based on the first ultrasound signal reflected from the anatomy of the subject and a second return signal based on the second ultrasound signal reflected from the anatomy of the subject. The received return signal is decoded based on a combination of the first return signal and the second return signal.

Example 85: The method of example 84, where the first return signal is combined with the second return signal based on a summation, an average, a weighted average, a cost-function-based combination, or a machine-learning-based combination.

Example 86: The method of example 57, where the transmission of the two or more ultrasound signals includes one or more asymmetric transmission characteristics.

Example 87: The method of example 86, where the one or more asymmetric transmission characteristics for the two or more ultrasound signals include one or more of a depth, a time, a rate, or an angle of probing of the anatomy of the subject.

Example 88: The method of example 86, where the one or more asymmetric characteristics are based on a presence of a contrast element within the anatomy of the subject.

Example 89: The method of example 86, further including transmitting, by at least one of the two or more transducer arrays, at least one of the two or more ultrasound signals at an area of the subject outside of a region of interest (ROI) and receiving, by the at least one of the two or more transducer arrays, an outside return signal based on the at least one of the two or more ultrasound signals reflected from the area of the subject outside of the ROI. The output is further generated based on the outside return signal and further includes information corresponding to the at least one of the two or more ultrasound signals reflected from the area of the subject outside of the ROI.

Example 90: The method of example 86, where the one or more asymmetric characteristics are based on a shape of an ultrasound field. The ultrasound field includes the two or more transmitted ultrasound signals.

Example 91: The method of example 86, where the one or more asymmetric characteristics are determined, by the one or more processors, using a machine-learned model.

Example 92: The method of example 57, further including generating, by the two or more transducer arrays, one or more depth ultrasound signals, the one or more depth ultrasound signals configured to determine a depth of penetration into the anatomy of the subject. The method further includes transmitting, by at least one of the two or more transducer arrays, the one or more depth ultrasound signals at the anatomy of the subject and receiving, by the at least one of the two or more transducer arrays, one or more depth return signals, the one or more depth return signals based on the one or more depth ultrasound signals reflected from the anatomy of the subject. The generation of the two or more ultrasound signals is based on the one or more depth return signals.

Example 93: An ultrasound device including a multi-array ultrasound scanner, one or more processors in communication with the multi-array ultrasound scanner, and a memory storing instructions that, when accessed by the one or more processors, cause the one or more processors to execute any one of the methods 57 through 92.

Example 94: A non-transitory, computer-readable media storing instructions that, when accessed by one or more processors, cause the one or more processors to execute any one of the methods 57 through 92.

Example 95: A computer program product storing instructions that, when accessed by one or more processors, cause the one or more processors to execute any one of the methods 57 through 92.

### CONCLUSION

Embodiments of multi-depth ultrasound imaging with a multi-frequency probe as described herein are advantageous, as they provide for one or more of increased resolution, targeted resolution, increased SNR, less scanning time for a patient, lower resource utilization for an associated ultrasound machine/system, and other benefits. The techniques of multi-depth ultrasound imaging with a multi-frequency probe disclosed herein also increase frame rates by employing a multi-line rolling buffer method, such as by buffering multiple pulse outputs and combining the pulse output response signals where they overlap in the anatomy being scanned. The multi-depth ultrasound imaging with a multi-frequency probe provides increased scanning efficiency, improved patient experience, higher-fidelity scanning outcomes, and similar benefits.

While the present subject matter has been described in detail with respect to various specific example implementations thereof, each example is provided by way of explanation and not limitation of the disclosure. Those skilled in the art, upon attaining an understanding of the foregoing, can readily produce alterations to, variations of, and equivalents to such implementations. Accordingly, the subject disclosure does not preclude inclusion of such modifications, variations, and/or additions to the present subject matter as would be readily apparent to one of ordinary skill in the art. For instance, features illustrated or described as part of one implementation can be used with another implementation to yield a still further implementation. Thus, it is intended that the present disclosure cover such alterations, variations, and equivalents.

As used herein, a phrase referring to “at least one of” a list of items refers to any combination of those items, including single members. As an example, “at least one of: a, b, or c” is intended to cover a, b, c, a-b, a-c, b-c, and a-b-c, as well as any combination with multiples of the same element (a-a, a-a-a, a-a-b, a-a-c, a-b-b, a-c-c, b-b, b-b-b, b-b-c, c-c, and c-c-c or any other ordering of a, b, and c).

While various embodiments of the disclosure are described in the foregoing description and shown in the drawings, it is to be distinctly understood that this disclosure is not limited thereto but may be variously embodied to practice within the scope of the following claims. From the foregoing description, it will be apparent that various changes may be made without departing from the spirit and scope of the disclosure as defined by the following claims.

1. An ultrasound device comprising:
a multi-array ultrasound scanner comprising two or more transducer arrays, the multi-array ultrasound scanner configured to:
generate, using the two or more transducer arrays, two or more ultrasound signals comprising two or more different frequencies, the two or more different frequencies configured to access two or more different depths in a subject;
encode the two or more ultrasound signals;
transmit the two or more encoded ultrasound signals at the two or more different depths in an anatomy of the subject; and
receive a return signal based on the two or more encoded ultrasound signals reflected from the two or more different depths in the anatomy of the subject;
one or more processors; and
a memory, the memory storing instructions that, when executed by the one or more processors, cause the one or more processors to generate an output, the output based on:
the received return signal; and
the two or more different frequencies.

2. The ultrasound device of claim 1, wherein the return signal is two or more return signals, and wherein at least one of the two or more return signals is based on a different one of the two or more encoded ultrasound signals reflected from the two or more different depths in the anatomy of the subject than at least one other of the two or more return signals.

3. The ultrasound device of claim 1, wherein the output is a combination image and the instructions further cause the one or more processors to:
generate at least first image data and second image data, the first image data based on a first frequency of the two or more different frequencies and the second image data based on a second frequency of the two or more different frequencies that is different from the first frequency; and
combine the first image data and the second image data to generate the combination image.

4. The ultrasound device of claim 3, wherein the instructions further cause the one or more processors to generate at least:
a first output image based on the first image data; and
a second output image based on the second image data, wherein the first image data and the second image data are combined to generate the combined image based on the first output image and the second output image.

5. (canceled)

6. The ultrasound device of claim 1, wherein the output is generated by the one or more processors using a machine-learned model, wherein at least the received return signal is provided as an input to the machine-learned model.

7. The ultrasound device of claim 1, wherein the return signal is a first return signal based on at least a first frequency of the two or more different frequencies associated with the two or more encoded ultrasound signals reflected from the anatomy of the subject, wherein the instructions further cause the one or more processors to:
receive a second return signal, the second return signal based on:
the two or more encoded ultrasound signals reflected from the anatomy of the subject; and
at least a second frequency of the two or more different frequencies associated with the two or more encoded ultrasound signals reflected from the anatomy of the subject, the second frequency being different than the first frequency;
generate, based on the first return signal and the first frequency, a first image; and
generate, based on the second return signal and the second frequency, a second image, wherein the output is further based on the first image and the second image.

8. A user interface (UI) for an ultrasound device, the UI configured to register one or more user inputs, which cause one or more processors to:
generate, by two or more transducer arrays of a multi-array ultrasound scanner, two or more ultrasound signals, the two or more ultrasound signals comprising two or more different frequencies, the two or more frequencies configured to access two or more different depths in a subject;
encode the two or more ultrasound signals;
transmit, using the two or more transducer arrays, the two or more encoded ultrasound signals at the two or more different depths in an anatomy of the subject;
receive, using the two or more transducer arrays, a return signal based on the two or more encoded ultrasound signals reflected from the two or more different depths in the anatomy of the subject; and
generate an output, the output based on:
the received return signal; and
at least two of the two or more different frequencies.

9. The UI of claim 8, wherein the return signal is two or more return signals, and wherein at least one of the two or more return signals is based on a different one of the two or more encoded ultrasound signals reflected from the two or more different depths in the anatomy of the subject than at least one other of the two or more return signals.

10. The UI of claim 8, wherein the output is generated by the one or more processors using a machine-learned model, wherein at least the received return signal is provided as an input to the machine-learned model.

11. (canceled)

12. The UI of claim 11, wherein:
the one or more user inputs further cause the one or more processors to determine an attention of an operator of the ultrasound device; and
the ROI is selected based on the attention of the operator.

13. The UI of claim 8, wherein the one or more user inputs further cause the one or more processors to determine, based on the generated output, a feature of interest within the anatomy of the subject.

14. The UI of claim 13, wherein the feature of interest is determined using a machine-learned model.

15. A method for multi-array ultrasound imaging, the method comprising:
generating, using two or more transducer arrays of a multi-array ultrasound scanner, two or more ultrasound signals comprising two or more different frequencies, the two or more frequencies configured to access two or more different depths in a subject;
encoding the two or more ultrasound signals;
transmitting, using the two or more transducer arrays, the two or more encoded ultrasound signals at the two or more different depths in an anatomy of a subject;
receiving, using the two or more transducer arrays, a return signal based on the two or more encoded ultrasound signals reflected from the two or more different depths in the anatomy of the subject; and
generating, by one or more processors, an output, the output based on:
the received return signal; and
the two or more different frequencies.

16. (canceled)

17. The method of claim 15, wherein the output is a combination image and the method further comprises:
generating at least a first image data and a second image data, the first image data generated based on a first frequency of the two or more different frequencies and the second image data generated based on a second frequency of the two or more different frequencies that is different than the first frequency; and
combining the first image data and the second image data to generate the combination image.

18. The method of claim 17, further comprising generating, by the one or more processors, at least:
a first output image based on the first image data; and
a second output image based on the second image data, wherein the first image data and the second image data are combined to generate the combined image based on the first output image and the second output image.

19. The method of claim 18, wherein the first output image and the second output image are combined to generate the combination image based on a combination of one or more pixels from the first output image with one or more pixels from the second output image.

20. The method of claim 19, wherein the one or more pixels from the first output image are combined with the one or more pixels from the second output image based on a summation, an average, a weighted average, a cost-function-based combination, or a machine-learning-based combination.

21. The ultrasound device of claim 1, wherein the encoding of the two or more ultrasound signals comprises a mathematical transformation operation.

22. The ultrasound device of claim 21, wherein:
the two or more ultrasound signals comprise two or more vectors; and
the mathematical transformation operation comprises one or more of:
a multiplication of one or more matrix operators on the two or more vectors; or
a type of encoding.

23. The ultrasound device of claim 22, wherein:
the matrix operator is a Hadamard matrix; or
the type of the encoding is one or more of a phase-inversion and a spatial rolling encoding.
